(19) United States
(12) Patent Application Publication
(10) Pub. No.: US 2025/0066269 A1
(43) Pub. Date: Feb. 27, 2025

(54) METHOD OF COMBINED SEPARATION AND CONVERSION OF AN OXYGENATE AND MICROCHANNEL REACTIVE DISTILLATION APPARATUS

(71) Applicant: Battelle Memorial Institute, Richland, WA (US)

(72) Inventors: Robert A. Dagle, Richland, WA (US); Johnny Saavedra Lopez, Richland, WA (US); Ward E. TeGrotenhuis, Kennewick, WA (US); Richard F. Zheng, Richland, WA (US); Dan R. Bottenus, Richland, WA (US); Paul H. Humble, Richland, WA (US); Stephen D. Davidson, Richland, WA (US)

(21) Appl. No.: 18/453,280

(22) Filed: Aug. 21, 2023

(51) Int. Cl. C07C 1/24; B01D 3/00; B01J 19/00; C10L 1/04

(52) U.S. Cl. CPC C07C 1/24; B01D 3/009; B01J 19/0093; C10L 1/04; C07C 2529/40

(57) ABSTRACT

A reactive distillation method, system, and apparatus are described for a combined separation and conversion of an oxygenate(s) in aqueous solution to more volatile intermediates for gas phase conversion. A mixture of oxygenate and water flow into a microchannel apparatus comprising a hydrophilic wick adjacent a reaction channel comprising a hydrophobic solid acid catalyst. During operation, water is produced in a dehydration reaction, produced water vapor is condensed away from the reaction channel and travels out through the wick layer, thus driving equilibrium toward more product. The combination of microchannel architecture with the catalytic distillation reaction was characterized by unexpectedly superior stability.

### STATEMENT REGARDING FEDERAL FUNDING

This work was financially supported by the U.S. Department of Energy (DOE), Office of Energy Efficiency and Renewable Energy (EERE), Bioenergy Technologies Office (BETO), and the Office of Technology Transitions (OTT) Technology Commercialization Fund (TCF). Work was performed at Pacific Northwest National Laboratory (PNNL) under Contract DE-AC05-76RL01830 and the National Renewable Energy Laboratory under Contract DE-AC36-08GO28308. The government has certain rights in the invention.

### INTRODUCTION

A challenge facing most bioprocessing operations is that hydrocarbon feedstock streams are diluted in water, thus requiring energy-intensive separations [1, 2]. Further, low product yields often are limited thermodynamically, not kinetically [3]. These challenges are coupled with the fact that biomass transportation costs drive the need for smaller, distributed processing plants. To incorporate the smaller scales desirable for biomass processing, novel processes with reduced capital costs must be developed. Microchannel reactive distillation (MCRD) has been identified as a process intensification technology that could provide energy savings while allowing downward scalability, thereby enabling processing at the scale relevant for biomass [4-6].

Conversion of fermentation-derived wet ethanol feedstock to hydrocarbon fuels is currently performed using process that are carbon efficient but energy and capital intensive. Dilute ethanol is first distilled from water, purified, and then dehydrated to ethylene. The ethylene then is oligomerized into a liquid hydrocarbon mixture that is separated into gasoline-, diesel-, and/or jet-boiling ranges after hydrogenation [see FIG. 1(A)][7]. Drawbacks to this approach include:
1. A significant amount of energy is required to first distill the ethanol/water mixture to 85-95% ethanol, which is necessary because conventional γ-Al₂O₃ dehydration catalysts are inhibited by water.
2. A significant amount of energy also is required to enable the highly endothermic ethanol dehydration reaction (+44.9 KJ/mol).
3. There exists a thermal mismatch between ethanol dehydration (˜350° C.) and oligomerization (<260° C.), requiring additional thermal energy to heat the reactor for dehydration and then cool down for oligomerization.

Significant amount of electrical power is required to compress low-pressure ethylene gas to high pressure for oligomerization. With MCRD technology, liquid ethanol is compressed, which consumes less energy than compressing ethylene gas.

Reactive distillation offers the potential for process intensification by reducing three unit operations to one operation (see FIG. 1) and lowering operating costs through energy savings. In conventional reactive distillation applied to ethanol dehydration, the heads of distillation columns almost exclusively process ethylene while the stripping and enriching sections of the columns are used for ethanol and diethyl ether (DEE) dehydration, respectively [8]. The use of microchannel technology, which provides rapid heat and mass transport due to the thin size of the channels, offers the potential for further process intensification [9].

The modular nature of microchannel technology also offers the potential for reduced capital costs via mass production manufacturing techniques.

Microchannel process technology increases the efficiency, effectiveness, and productivity of chemical reactors in which reaction rates can be accelerated up to 1,000× faster than conventional systems. This improvement is made possible because the passages are dramatically smaller than those in conventional systems [9].

Chemical reactions generally are limited by heat and mass transfer performance. For this reason, microchannel technology has the potential to greatly improve overall system performance for a wide range of chemical industry applications.

The net result is an overall 10× system volume reduction or more compared to conventional hardware, increased product yield, improved energy efficiency, and enabling of novel reaction pathways [9]. The benefits of microchannel reactors have been demonstrated for many reactions including stream reforming [10, 11] and Fischer-Tropsch synthesis [9, 12] applications.

We have previously demonstrated microchannel distillation (MCD) at the laboratory scale for separation processes such as fractionation of JP-8 diesel fuel [13], purification of trace contaminants from n-octane [13], separation of propane/propylene [14], isotopic enrichment of methane [15], and air separation [16]. The height of a theoretical plate (HETP) has been reduced to less than 0.5 cm [15], where the best available technologies are 2 cm HETP with Sulzer columns (<8 cm diameter) and ˜1 ft for conventional large-diameter reactors [17]. Thus, a 60× reduction in HETP has been demonstrated for MCD relative to conventional distillation technology [15]. Furthermore, modular MCRD scales up linearly, and capillary liquid flow avoids the need for tall columns that hinder distributed processing. Additionally, heat exchange can be incorporated for large heats of reaction. This modular approach is particularly appealing for distributed processing applications where challenges include high capital costs, low economies of scale, and dilute aqueous streams that make separations energy intensive.

The reaction of ethanol to ethylene and water is endothermic and typically carried out at mild temperatures [e.g., 250-350° C., Eqn. (1)] [18]. At lower temperatures (e.g., 200-250° C.), dehydration to DEE is thermodynamically favored [Eqn. (2)] [18]. DEE can be converted to both ethanol and ethylene, which is favored at mild temperatures and lower pressures [Eqn. (3)] [18, 19]. Taken together, ethylene production is favored at mild temperatures, low pressures, and with lower water content reactants. The ethanol dehydration reaction to ethylene proceeds by an elimination mechanism via decomposition of ethoxy groups over solid acid catalysts [18]. Lower temperature offers a consecutive path via DEE formation and subsequent conversion to ethylene. Higher temperatures are required for selective conversion to ethylene (see Scheme 1).

With reactive distillation, the dehydration reaction equilibrium shifts towards completion by immediate removal of the water byproduct upon formation, while maintaining an aqueous feedstock in the condensed phase. This enables the use of lower operating temperatures than would be possible if simultaneous separation and conversion was not performed. By using microchannels, the potential for further process intensification exists.

### SUMMARY OF INVENTION

We have developed a method for the combined separation and conversion of an oxygenate(s) in aqueous solution to more volatile intermediates for gas phase conversion.

In one aspect, the invention provides a process for alcohol dehydration by reactive distillation and immediate removal of the water byproduct upon formation, using microchannels. In another aspect, the invention provides a novel design of a microchannel device for reactive distillation. The invention also includes systems that include apparatus, compositions, and/or conditions such as temperature and pressure.

In another aspect, the invention provides apparatus for the combined separation and conversion of an oxygenate in aqueous solution to more volatile intermediates for gas phase conversion, comprising: a liquid inlet connected to a wicking microchannel; an outlet connected to the wicking microchannel; a vapor microchannel adjacent the wicking microchannel; a vapor inlet connected to the vapor microchannel and a vapor outlet connected to the vapor microchannel; a hydrophilic wick disposed in the wicking microchannel; and a hydrophobic solid acid catalyst disposed in the vapor microchannel.

In another aspect, the invention provides a method of combined separation and conversion of an oxygenate(s) in aqueous solution to more volatile intermediates for gas phase conversion, comprising: passing a liquid comprising between 1 and 80 wt. % oxygenates and 20 to 99 wt. % water into a reaction chamber; wherein the reaction chamber comprises a wicking microchannel comprising a hydrophilic porous wick for liquid transport, and a vapor microchannel adjacent the wick for gas phase conversion having a thickness of 1 cm or less; wherein the vapor microchannel comprises a heterogenous catalyst; heating the liquid in the wicking microchannel wherein oxygenate is stripped from the liquid phase and dehydrated over a hydrophobic solid acid catalyst to produce water and a gaseous dehydrated product, and wherein the produced water preferentially passes back into the aqueous solution in the wick; wherein the aqueous solution passes through the wick and out of the reaction chamber via a first liquid outlet;

and wherein the gaseous dehydrated product passes through the vapor microchannel and out of the reaction chamber through a vapor outlet.

In a further aspect, the invention provides a system comprising;
a liquid comprising between 1 and 80 wt. % oxygenates and 20 to 99 wt. % water in a reaction chamber; wherein the reaction chamber comprises a wicking microchannel comprising a hydrophilic porous wick for liquid transport, and a vapor microchannel adjacent the wick for gas phase conversion having a thickness of 1 cm or less; wherein the vapor microchannel comprises a heterogenous catalyst; a liquid in the wicking microchannel wherein oxygenate is stripped from the liquid phase and dehydrated over a hydrophobic solid acid catalyst to produce water and a gaseous dehydrated product, and wherein the produced water preferentially passes back into the aqueous solution in the wick;
wherein the aqueous solution passes through the wick and out of the reaction chamber via a first liquid outlet; and wherein the gaseous dehydrated product passes through the vapor microchannel and out of the reaction chamber through a vapor outlet.

Preferably, the system comprises a first heat exchange channel in contact with the wicking microchannel and a second heat exchange channel in contact with the vapor microchannel; wherein the first heat exchange channel in contact with the wicking microchannel has a lower temperature than the second heat exchange channel in contact with the vapor microchannel. A system comprises apparatus plus fluids and conditions within the apparatus.

In some preferred aspects, the hydrophilic wick comprises a FeCrAlY foam. This is a surprising feature of this aspect of the invention since FeCrAlY foam was reported to be a hydrophobic material (Zhang et al Appl. Surf. Sci. 276 (2013) 377-382).

The MCD platform technology, through integration of catalytic material in the vapor channel adjacent to the wick, provides energy savings, process intensification, and compact modular reactors, ultimately providing reductions to both capital and operating costs. Microchannel reactive distillation technology can reduce operating and capital costs by at least 35% and 55%, respectively, relative to the incumbent alcohol-to-jet technology.

The invention (or the resistive heating layer) may also be further characterized by having one or any combination of the properties (or within ±30% or ±20% or ±10% or ±5%) of one or any combination of the properties described herein including in the Examples. For example, any aspect of the invention can be further characterized by the ethanol (or oxygenate) conversion (or within ±30% or ±20% or ±10% or ±5% of the ethanol (or oxygenate) conversion) at the conditions described in the examples.

Various aspects of the invention are described using the term “comprising;” however, in narrower embodiments, the invention may alternatively be described using the terms “consisting essentially of” or, more narrowly, “consisting of.”

### Glossary

A “capture structure” is a structure disposed (at least partly) within a gas flow channel that assists movement of a liquid into the wick.

Conversion—The term “conversion of a reactant” refers to the reactant mole or mass change between a material flowing into a reactor and a material flowing out of the reactor divided by the moles or mass of reactant in the material flowing into the reactor. For example, if 100 grams of ethanol are fed to a reactor and 30 grams of ethanol are flowing out of the reactor, the conversion is [(100−30)/100]=70% conversion of ethanol.

The terms “hydrophilic” and “hydrophobic” are relative terms. The wick is hydrophilic so that under the conditions of the separation, an aqueous stream flows through the wick. The reaction channel (vapor channel) is hydrophobic so that, under the conditions of the separation, an aqueous stream does not flow through the reaction channel. A droplet of water present in the vapor channel and in contact with the wick will be drawn into the wick. This is the conventional understanding of “hydrophilic” and “hydrophobic” materials. In a preferred definition, hydrophilic materials have a water contact angle less than 90 degrees and hydrophobic materials have a water contact angle greater than 90 degrees as described (for non-foam materials) in “Definitions for Hydrophilicity, Hydrophobicity, and Superhydrophobicity: Getting the Basics Right,” J Phys Chem Lett. 2014 Feb. 20; 5 (4): 686-8. For foam or felt materials, contact angle is measured as described in Zhang et al. Appl. Surf. Sci. 276 (2013) 377-382.

A “laminated device” is a device having at least two nonidentical layers, wherein these at least two nonidentical layers can perform a unit operation, such as heat transfer, desorption, etc, and where each of the two nonidentical layers are capable having a fluid flow through the layer. In the present invention, a laminated device is not a bundle of fibers in a fluid medium. In preferred embodiments, a laminated device is formed from at least 10 sheets, with each sheet having a thickness of less than 1 cm, preferably less than 5 mm.

A “liquid” is a substance that is in the liquid phase within the wick under the relevant operating conditions.

A “liquid flow channel” is a wick (or wicks) or open channel (or channels) or a combination of wicks and open channels through which a liquid flows during operation of a device.

A “microchannel” is a channel having at least one internal dimension of 10 mm or less, preferably 2 mm or less, and greater than 1 μm (preferably greater than 10 μm), and in some embodiments 50 to 500 μm; preferably a microchannel remains within these dimensions for a length of at least 1 cm, preferably at least 20 cm. In some embodiments, in the range of 5 to 100 cm in length, and in some embodiments in the range of 10 to 60 cm. Microchannels are also defined by the presence of at least one inlet that is distinct from at least one outlet. Microchannels are not merely channels through zeolites or mesoporous materials. The length of a microchannel corresponds to the direction of flow through the microchannel. Microchannel height and width are substantially perpendicular to the direction of flow of through the channel. In the case of a laminated device where a microchannel has two major surfaces (for example, surfaces formed by stacked and bonded sheets), the height is the distance from major surface to major surface and width is perpendicular to height. In preferred embodiments of this invention, microchannels are straight or substantially straight-meaning that a straight unobstructed line can be drawn through the microchannel (“unobstructed” means prior to particulate loading). Typically, devices comprise multiple microchannels that share a common header and a common footer. Although some devices have a single header and single footer; a microchannel device can have multiple headers and multiple footers. In this invention, the wick microchannel and reaction microchannel are adjacent so reactants and products travel directly from one microchannel to the other, typically directly adjacent although in some embodiments a porous screen can be interposed between reaction and wicking microchannels.

Heat exchange fluids may flow through heat transfer microchannels adjacent to process channels (preferably reaction microchannels), and can be gases or liquids and may include steam, liquid metals, or any other known heat exchange fluids—the system can be optimized to have a phase change in the heat exchanger. In some preferred embodiments, multiple heat exchange layers are interleaved with multiple reaction microchannels. For example, at least 10 heat exchangers interleaved with at least 10 reaction microchannels and preferably there are 10 layers of heat exchange microchannel arrays interfaced with at least 10 layers of reaction microchannels. Each of these layers may contain simple, straight channels or channels within a layer may have more complex geometries.

“Oxygenates” are compounds that contain carbon and oxygen atoms. Non-limiting classes of oxygenates include alcohols, carboxylic acids, aldehydes, ketones, and esters. Some specific examples include methanol, ethanol, propanol, butanol, formic acid, acetic acid, propionic acid, formaldehyde, acetaldehyde, propionaldehyde, and acetone. Pore size-Pore size relates to the size of a molecule or atom that can penetrate into the pores of a material. As used herein, the term “pore size” for zeolites and similar catalyst compositions refers to the Norman radii adjusted pore size well known to those skilled in the art. Determination of Norman radii adjusted pore size is described, for example, in Cook, M.; Conner, W. C., “How big are the pores of zeolites?” Proceedings of the International Zeolite Conference, 12th, Baltimore, Jul. 5-10, 1998; (1999), 1, pp 409-414. Generally, for non-zeolite materials, “pore size” is volume average pore size. This can be measured by known techniques such as optical or electron microscopy, or mercury porosimetry.

One of ordinary skill in the art will understand how to determine the pore size (e.g., minimum pore size, average of minimum pore sizes) in a catalyst. For example, x-ray diffraction (XRD) can be used to determine atomic coordinates. XRD techniques for the determination of pore size are described, for example, in Pecharsky, V. K. et at, “Fundamentals of Powder Diffraction and Structural Characterization of Materials,” Springer Science+Business Media, Inc., New York, 2005. Other techniques that may be useful in determining pore sizes (e.g., zeolite pore sizes) include, for example, helium pycnometry or low-pressure argon adsorption techniques. These and other techniques are described in Magee, J. S. et at, “Fluid Catalytic Cracking: Science and Technology,” Elsevier Publishing Company, Jul. 1, 1993, pp. 185-195. Pore sizes of mesoporous catalysts may be determined using, for example, nitrogen adsorption techniques, as described in Gregg, S. J. at al, “Adsorption, Surface Area and Porosity,” 2nd Ed., Academic Press Inc., New York, 1982 and Rouquerol, F. et al, “Adsorption by powders and porous materials. Principles, Methodology and Applications,” Academic Press Inc., New York, 1998.

The term “preferentially” has the conventional meaning that water preferentially partitions into the wicking channel as compared to the vapor channel.

Selectivity—The term “selectivity” refers to the amount of production of a particular product in comparison to a selection of products. Selectivity to a product may be calculated by dividing the amount of the particular product by the amount of a number of products produced. For example, if 75 grams of products are produced in a reaction and 20 grams of ethylene are found in these products, the selectivity to ethylene amongst products is 20/75=26.7%. Selectivity can be calculated on a mass basis, as in the aforementioned example, or it can be calculated on a carbon basis, where the selectivity is calculated by dividing the amount of carbon that is found in a particular product by the amount of carbon that is found in a selection of products. Unless specified otherwise, selectivity is on a mass basis. For reactions involving conversion of a specific molecular reactant (ethanol, for example), selectivity is the percentage (on a mass basis unless specified otherwise) of a selected product divided by all the products produced.

A “solid acid catalyst” is a solid under the intended reaction conditions. In the present invention, the catalyst can catalyze dehydration of an alcohol. As is known, solid acid catalysts can be characterized by Hammett titration. Preferably, the catalyst is a ZSM-5 zeolite catalyst. Other types of zeolite catalysts include: ferrierite, zeolite Y, zeolite beta, mordenite, MCM-22, ZSM-23, ZSM-57, SUZ-4, EU-1, ZSM-11, (S) AIPO-31, SSZ-23, among others. In other embodiments, non-zeolite catalysts may be used; for example, WOx/ZrO2, aluminum phosphates, metal-organic frameworks, etc. In some embodiments, the catalyst may comprise a metal and/or a metal oxide. Suitable metals and/or oxides include, for example, nickel, palladium, platinum, titanium, vanadium chromium, manganese, iron, cobalt, zinc, copper, gallium, and/or any of their oxides, among others. In some preferred embodiments, the acid is a Bronsted acid. Properties of the catalysts (e.g., pore structure, type and/or number of acid sites, etc.) may be chosen to selectively produce a desired product.

### BRIEF DESCRIPTION OF DRAWINGS

FIG. 1A-B: Comparison between conventional alcohol dehydration and method proposed in this invention A) Conventional alcohol-to-jet technology utilizing ethanol dehydration to ethylene and oligomerization to jet-range hydrocarbons. (B) Proposed process replacing the rectifier, molecular sieve and ethylene reactor with microchannel reactive distillation.

FIG. 2: Schematic illustration of a microchannel device used in the examples. Two Hastelloy halves (top and bottom) were assembled using hexagonal screws and sealed using elastomer O-rings (e.g., silicone, kalrez, viton, PTFE) set in a groove. Liquid fills the inlet head channel and is transported to the outlet channel through the wick (dimensions 9.1×4.5 cm) and variable thickness (up to 2 mm). Catalyst (0-2 g) was contained in the vapor channel.

FIG. 3A-3B: Experimental set-up for the MCRD device. FIG. 3A shows the vapor channel and liquid channel that constitute the reaction chamber. Inlets and outlets are also shown. FIG. 3B schematically illustrates the system.

FIG. 4A-4B: Evaluation of FeCrAlY felt for the transport of ethanol/water mixtures inside MCRD device. FIG. 4A (top) shows the flow pattern used in the microchannel device. In this device, the flow rate of liquid leaving the column is controlled by the height difference with respect to the liquid channel inside the device (ΔH). FIG. 4B (bottom) shows the fraction of liquid pulled from the wick as a function of the outlet column height (ΔH) for 2 different flows (3 and 10 mL/min). The wick was a modified FeCrAlY felt (Technetics, 19%, 19% density, 0.01 in. thickness).

FIG. 5: Hydrostatic pull test using the FeCrAlY wick used in the MCRD device development. To test the stability of the siphon in the wick, the system was completely flooded, and the liquid drained from the device was monitored at variable siphon heights (in 0.5 cm increments). As the outlet was further lowered, more liquid drained from the device. FIG. 5 shows 3 different regimes: first, the excess liquid in vapor channel was drained with no major resistance (ΔH<1.5 cm), then (from DH 1.5-4.5 cm) the amount of liquid remained constant, indicating that the liquid is being retained by the wick, finally as enough pull is created the remaining liquid from the device is evacuated. The ΔH 4.5 cm column height necessary to break the siphon, indicates that the siphon can withstand disturbances of ˜0.064 psi. Based on the hydrostatic siphon test, a ΔH of 3 cm for the driving column would provide sufficient pull to drain the vapor channels while not over stressing the siphon across the wick.

FIG. 6A-6B: Evaluation of gas phase catalyst performance stability vs time on stream. Ethanol dehydration catalyst stability A) HZSM-5 (Si/Al=23) B) 3 wt. % Triflic Acid/HZSM-5 (Si/Al=23). Both: temperature 225° C.; WHSV 3.22 h⁻¹; pressure 200 psi.

FIG. 7A-7B: Vapor-liquid equilibrium calculations for mixtures N2/EtOH/H2O. A) P-T plot showing Bubble point for mixtures with different moisture molar contents. B) P-T plot showing the Bubble/Dew point limits for 40% w EtOH/H2O mixture. Calculations assume Raoult's law using Antoine's vapor pressure.

FIG. 8: ChemCAD modelling calculations showing the equilibrium ethanol/water conversion and selectivity to ethylene or diethyl ether as a function of pressure and temperature.

FIG. 9A-9B: Results for the parametric study of HZSM5 used in the gas phase ethanol dehydration reaction over CBV2314 catalyst. A) conversion vs reaction temperature (GHSV=5000 h-1), B) Selectivity changes (diethyl ether, ethylene, acetaldehyde) vs reaction temperature.

FIG. 10A-10B: Ethanol conversion (A—left) and Ethylene selectivity (B—right) vs GHSV at two different total pressure (atmospheric and 410 psi). Catalyst: CB2314 HZSM5, Reaction Temperature: 250° C.

FIG. 11: Ethanol conversion and ethylene selectivity as a function of time-on-stream. Catalyst: CB2314 HZSM5, Reaction Temperature: 250° C. Catalyst Stability. 225° C., 200 psi, 5000 h⁻¹.

FIG. 12: RMCD catalytic performance test results as a function of temperature, pressure, and weight-hour-space-velocity.

### DETAILED DESCRIPTION OF INVENTION

The apparatus of the invention comprises a wicking microchannel adjacent to a vapor microchannel. Preferably, there is a liquid inlet connected to one end of the wick (in the length direction) and a liquid outlet connected to another end of the wick (in the length direction). In operation, the vapor phase is contiguously connected to the vapor outlet and the liquid phase is contiguously connected from the liquid flow path to the liquid outlet.

The apparatus is preferably a layered structure with a wicking layer adjacent a vapor layer. Each layer is no more than 1 cm thick and a device may comprise a plurality of layers. Each layer can be a contiguous layer with length and width each at least ten times greater than thickness; alternatively, each layer may be comprised of a parallel array of microchannels with length at least ten times greater than both thickness and width.

Preferably, the gas flow channels and/or liquid flow channels are essentially planar in the fluid separation regions This configuration enables highly rapid and uniform rates of mass and heat transport. In some preferred embodiments, the gas flow channels and/or liquid flow channels have dimensions of width and length that are at least 10 times larger than the dimension of thickness (which is perpendicular to net gas flow). In especially preferred embodiments, the devices are made by stacking planar shims (plates) and bonding the stacked shims. Preferably, the shims are less than 1 cm thick, more preferably less than 5 mm thick. In some preferred embodiments there are multiple gas flow channels operating in parallel. This configuration allows high throughput and provides a large surface area to volume ratio for high efficiency. In some preferred embodiments, layers are stacked to have between 2 and 600 layers of gas flow channels, more preferably at least 3 layers of gas flow channels, and in some embodiments, between 3 and 40 layer of gas flow channels.

The apparatus is preferably configured with a heat source adjacent to the reaction channel and a cooling source adjacent the wicking channel. In some embodiments, a reaction chamber is in thermal contact with a microchannel heat exchanger. This combination of reaction chamber(s) and heat exchanger(s) can result in high rates of thermal transfer. Examples and more detailed description including the use of microchannel heat exchangers are provided in U.S. Pat. No. 6,616,909 incorporated herein by reference. In some embodiments, the reaction chamber(s) and heat exchangers have a heat flux of at least 0.1 W per cubic centimeter of reactor volume. General descriptions of microchannel architecture and separation of components include US U.S. Pat. Nos. 6,666,909; 7,610,775; 7,272,941; and 8,438,873 all of which are incorporated herein by reference as if reproduced in full below.

In the devices of the present invention, the primary heat transfer surfaces are the walls between the heat exchangers and the fluid flow paths. Walls between channels in a heat exchanger can act as heat exchange fins, and thus provide extended heat transfer surface area. Walls within a heat exchanger can also provide structural support. For good thermal transport, walls between layers are preferably 500 μm thick or less; in some embodiments in the range of 100 to 500 μm.

Fluid flowing through the heat exchanger channels can be a liquid (for example, water) or a gas. In some embodiments, a fan or blower moves gas through the cooling channels. In some preferred applications of the present invention, it is desired to use a gas as the heat exchange fluid. In this case, the majority of the heat transfer resistance can be in the heat exchange channel. In some embodiments, a configuration with an extended heat transfer surface in the beat exchange channels is preferred.

Cross-flow heat exchange can be used to provide for heat exchange fluid flow path and less pressure drop, however, in some preferred embodiments, the flow through the heat exchanger is counter-current flow and, in some other embodiments, co-flow For all the devices described herein, the shims can be repeated for numerous layers, and, in some embodiments, the devices include 2 to 1000, or at least 4, repeating units, where the repeating unit includes shims for desorption (including a wick and gas flow channel) and heat exchange.

A wick is a material that will preferentially retain a wetting fluid by capillary forces and through which there are multiple continuous channels through which liquids may travel by capillary flow. The channels can be regularly or irregularly shaped. Liquid will migrate through a dry wick, while liquid in a liquid-containing wick can be transported by applying a pressure differential, such as suction, to a part or parts of the wick. The capillary pore size in the wick can be selected based on the contact angle of the liquid and the intended pressure gradient in the device, and the surface tension of the liquid. Preferably, the pressure differential across the wick during operation should be less than the breakthrough pressure—the point at which gas will intrude into the wick displacing the liquid—this will exclude gas from the wick.

The wick could be a uniform material, a mixture of materials, a composite material, or a gradient material. For example, the wick could be graded by pore size or wettability to help drain liquid in a desired direction The wick materials are hydrophilic and porous. If a metal is used it should be oxidized to convert the surface to a hydrophilic surface. If a polymer is used as the wick, it should be insoluble and hydrophilic. Examples of wick materials suitable for use in the invention include: sintered metals. metal screens, metal foams, ceramic or polymeric foams, polymer fibers including cellulosic fibers, or other hydrophilic, porous materials. In some preferred embodiments, the wick comprises a screen, foam, or sintered metal. A screen, especially an oxidized metal screen is particularly preferred. The pore sizes in the wick materials are preferably in the range of 10 nm to 1 mm, more preferably 100 nm to 0.1 mm, where these sizes are the largest pore diameters in the cross-section of a wick observed by scanning electron microscopy (SEM). An especially preferred wick is FeCrAlY felt that is oxidized to form an alumina surface.

In operation of a device with a wick, the wick should not be flooded, and it is preferably not dry A wet or saturated wick will effectively transport liquid through capillary forces to a low pressure zone, such as low pressure created by suction or other means of creating a pressure differential. The inventive devices can be operated so that liquid flows in the direction of gravity, and gas can flow countercurrent—in this configuration, the devices can operate with or without suction.

A capture structure can be inserted (at least partly) within the gas flow channel, and in liquid contact with the wick. The capture structure assists in removing (capturing) a liquid from the gas stream. One example of a capture structure are cones that protrude from the wick; liquid can condense on the cones and migrate into the wick—an example of this capture structure is shown in U.S. Pat. No. 3,289,752, incorporated herein by reference Other capture structures include inverted cones, a liquid-nonwetting porous structure having a pore size gradient with pore sizes getting larger toward the wick, a liquid-wetting porous structure having a pore size gradient with pore sizes getting smaller toward the wick and fibers such as found in commercial demisters or filter media.

The apparatus may include additional layers or separate components. For example, the apparatus may comprise a reboiler connected to an outlet of the wicking microchannel. The reboiler collects the aqueous liquid from the wicking microchannel (or a plurality of wicking microchannels) and heats the aqueous solution. The heated aqueous solution can be recycled to a wicking microchannel.

Another optional feature is reduced or non-wettability of one or more walls in the gas flow channel. This could be accomplished, for example, by making this wall of, or coating the wall with, a hydrophobic material.

The vapor channel is a reaction channel that contains a solid acid catalyst. The solid acid catalyst may comprise, for example, a zeolite, mesoporous silica such as SBA15, silicoaluminum phosphate (SAPO), or a titania supported oxide. A particularly preferred catalyst is ZSM-5 having a Si/Al ratio of at least 15 or at least 20.

In some embodiments, catalysts are in the form of inserts that can be conveniently inserted and removed from a reaction chamber. Reaction chambers (either of the same type or of different types) can be combined in series with multiple types of catalysts. For example, reactants can be passed through a first reaction chamber containing a first type of catalyst, and the products from this chamber passed into a subsequent reaction chamber (or a subsequent stage of the same reaction chamber) containing a second type of catalyst in which the product (or more correctly termed, the intermediate) is converted to a more desired product. If desired, additional reactant(s) can be added to the subsequent reaction chamber.

The catalyst could also be applied by other methods such as wash coating. On metal surfaces, it is preferred to first apply a buffer layer by chemical vapor deposition, thermal oxidation, etc. which improves adhesion of subsequent wash coats.

Preferred apparatus and methods of conducting reactions and separations can be characterized by their properties. Unless specified otherwise, these properties are measured using the testing conditions described in the Examples section. The invention can be characterized by any of the properties individually or in any combination.

The devices may be made of materials such as plastic, metal, ceramic and composites, depending on the desired characteristics. Walls separating the device from the environment may be thermally insulating; however, the walls separating heat exchangers and adjacent reaction channels or wicking channels and should be thermally conductive.

Reactive distillation is known in conventional systems. For example, Terrill et al. in U.S. Pat. No. 9,114,328 describe a process for producing a stream of glycolate ester oligomers and glycolic acid oligomers while simultaneously removing water with reactive distillation. Kiss, et al., in “Reactive Distillation: Stepping Up to the Next Level of Process Intensification”, Ind. Eng. Chem. Res. (2019) 58, 15, 5909-5918 provide a review of conventional Reactive Distillation, and in section 3.5 gives examples of water removal to drive the reaction forward. Shah et al., in “Reactive Distillation: An Attractive Alternative for the Synthesis of Unsaturated Polyester”, Macromolecular Symposia, (2011) 302 (1), 46-55 describe conventional reactive distillation for the polyesterification reaction with water removal to push the reaction equilibrium.

The methods of the present invention apply to reactions in which water is liberated. Examples include dehydration of alcohol to olefins; conversion of carboxylic acids to ketones; and aldol condensation of carbonyls to form a conjugated enone.

### EXAMPLES

As an example, we demonstrated the proof-of-concept for microchannel reactive distillation (MCD) for alcohol-to-jet application: combining ethanol/water separation and ethanol dehydration in one unit operation. Ethanol is first distilled into the vapor phase, converted to ethylene and water, and then the water co-product is condensed to the shift reaction equilibrium. Process intensification is achieved through rapid mass transfer-ethanol stripping from thin wicks using novel microchannel architectures-leading to lower residence time and improved separation efficiency. The operation enables low temperatures (<250° C.) and mild pressures (<500 psig), which are compatible with the conditions necessary for the downstream ethylene oligomerization reaction. Energy savings are realized with integration of unit operations. For example, heat of condensing water can offset vaporizing ethanol. Furthermore, the dehydration reaction equilibrium shifts towards completion by immediate removal of the water byproduct upon formation while maintaining aqueous feedstock in the condensed phase. Conversion of 40% ethanol in water to ethylene was demonstrated with 91% ethylene selectivity and 71% ethanol conversion at 220° C., 600 psig, and 0.28 hr⁻¹. Almost three stages of separation (2.7) were also demonstrated, under these conditions, using a device length of 9.1 cm. This provides a height equivalent of a theoretical plate (HETP), a measure of separation efficiency, of ˜3.3. By comparison, conventional distillation packing provides an HETP of ˜30 cm. Thus, 9× reduction in HETP was demonstrated over conventional technology, providing a means for significant energy savings. Finally, preliminary process economic analysis indicated that by using microchannel reactive distillation technology the operating and capital costs could be reduced by at least 35% and 55%, respectively, relative to the incumbent alcohol-to-jet technology, provided future improvements to MCRD design and operability are made.

### Example I

### Experimental Methods

### Catalyst Synthesis

ZSM5 zeolite has been widely used as a standard catalyst for ethanol dehydration. In this study we used ZSM5 CBV2314 from Zeolyst (Si/A=23), its acidity was modified with different precursors: ammonium phosphate (P), triflic acid (TFA), polytungstic acid (PWA). The activity of these catalyst was compared to catalysts with predominantly Lewis acidity (Ag—W/Al₂O₃, Zr/SiO2, γ-Al₂O₃) and ZSM5 with increased Si/Al ratio CBV8014 (Si/Al=80). Catalyst screening was performed in the gas phase using 1.5 g of catalyst at 225° C., 200 psi, GHSV=5000 h⁻¹, with molar gas composition: 10.56% N2, 18.75% EtOH, 71.62% H₂O. Before reaction, catalysts were pre-treated overnight under N₂ (100 sccm).

CB2314 ZSM5 in the ammonium form was calcined at 500° C. for 4 h (5° C./min) to produce the material in the hydrogen form. 3 wt % of different modifiers were added from aqueous solutions according to synthesis methods reported in the literature. The ZSM5 solid was suspended and stirred in water solutions of the acid precursors: ammonium phosphate (P) [12], triflic acid (TFA) [13], polytungstic acid (PWA) [14]. After removing the water stirring at 60° C., the catalysts were completely dried at 120° C. overnight. Ammonium precursor was thermally decomposed by calcination at 500° C., the other catalysts were calcined at lower temperature (200° C.) due to its low thermal stability. Ag—W/Al2O3, Zr/SiO2 catalysts (3/1, and 20/1 mol/mol) were prepared using incipient wetness impregnation from silver nitrate and zirconyl nitrate water solutions over γ-Al₂O₃ (Engelhard) and SiO2. After impregnation, the catalysts were dried at 110° C. for 8 hours and calcined at 500° C. for 4 hours.

### Catalyst Performance Evaluations

The parametric studies were performed in the gas phase similarly to the catalyst screening experiments. A fixed amount of each catalyst was loaded to the reactor (1.5 g, 60-100 mesh, 0.5 in OD stainless steel reactor) and dried in-situ overnight under 100 sccm of N2 at 200° C. Using the same catalyst load, the reaction conditions were varied: temperature ranges from 150 to 280° C., pressure from atmospheric to 400 psi, GHSV from 1250 to 5000 h⁻¹. Catalyst stability was evaluated using three sequential 12 h long runs and deactivation was verified after each change in conditions.

### Microchannel Reactive Distillation Experimental Setup

The microchannel device was a Hastelloy device divided into two halves that are assembled using hexagonal bolts. Elastomer o-rings (silicone, kalrez, viton, PTFE) are used to seal the device. The lower channel is used to host the microwick (9 cm length) and transport liquid, the channel that feeds liquid to the device is flooded and distributes the liquid uniformly to the wick. FeCrAlY felt (Technetics) 19% density, 0.01 in thickness was used as wick. The upper channel is used to transport vapor/gas.

A 500D ISCO syringe pump is used to feed liquid to the device, Brooks MFC for nitrogen, the re-boiler and device are heated using heat tapes, the temperature is controlled using DigiSense temperature controllers. BPR Tescom 0-1000 psi for Pressure control, liquid is collected at 5° C., MicroGC and drycal for analysis and flow measurement.

The Fenske Equation (Eqn. 4) was used to calculate the theoretical number of stages of separation occurring in the MCRD during the separation test.

$$N = \frac{log [ ( \frac{{X}_{d}}{1 - {X}_{d}} ) ( \frac{1 - {X}_{b}}{{X}_{b}} ) ]}{log {α}_{a v g}} \qquad ( 4 )$$

Here N is the number of stages of separation. Xd is the mole fraction of the light key in the distillate and Xb is the mole fraction of the heavy key. Finally, α_{avg} is the average relative volatility of the light and heavy keys. The MCRD is not like a traditional distillation tower in that there are no discrete stages and it is instead performing continuous separations, more like a packed distillation tower. To calculate the height equivalent of a theoretical plate for the MCRD it is simply the characteristic length, L, of the wick (in this case 9 cm) divided by the number of stages calculated from the Fenske Equation, N (Eqn. 5).

$$HETP = \frac{L}{N} \qquad ( 5 )$$

### ChemCAD Reactor Modelling

Gas-Liquid calculations to obtain the bubble and dew point for mixtures ethanol/water were done using the Raoult's law and Antoine equations for the calculation of vapor pressure. ChemCAD was used to model the RMCD equilibrium. A ten-stage distillation tower was used with reactions enables in the bottom five stages (reboiler through tray 4). The equilibrium constant was calculated based on reaction temperature from the Equation 6 shown below and the values used for A, B, C, and D are shown in Table 1. Feed was set to deliver to Stage 5 (Tray 4) matching RMCD temperature and pressure. To converge the distillation tower, the two fixed parameters selected were Bottoms Temperature (matching RMCD operation) and Distillate Composition to be 0.01 mole fraction water. PSKR (Peng-Robinson and Soave-Redlich-Kwong) with regular SKR/PR BIPs was selected for the thermodynamic package. For direct comparison to the modeled RMCD system, a Gibbs reactor (Gibbs free energy minimization) under the same feed conditions was used to assess the reaction equilibrium alone.

$$ln ( {K}_{e q} ) = A + B / T + C ( ln ( T ) ) + D \cdot T \qquad ( 6 )$$

$$Et OH ↔ {H}_{2} O + {C}_{2} {H}_{4} \qquad ( 7 )$$

$$2 Et OH ↔ {H}_{2} O + {C}_{4} {H}_{8} O \qquad ( 8 )$$

TABLE 1
Equilibrium coefficient calculation values

| Eqn. | Reaction | A | B | C | D |
|---|---|---|---|---|---|
| 2 | Ethanol to Ethylene | 18.234 | −5261.8 | 0 | 0 |
| 3 | Ethanol to Diethyl | −2.5853 | 2460.5 | 0 | 0 |
| | Ether | | | | |

### Technoeconomic Analysis

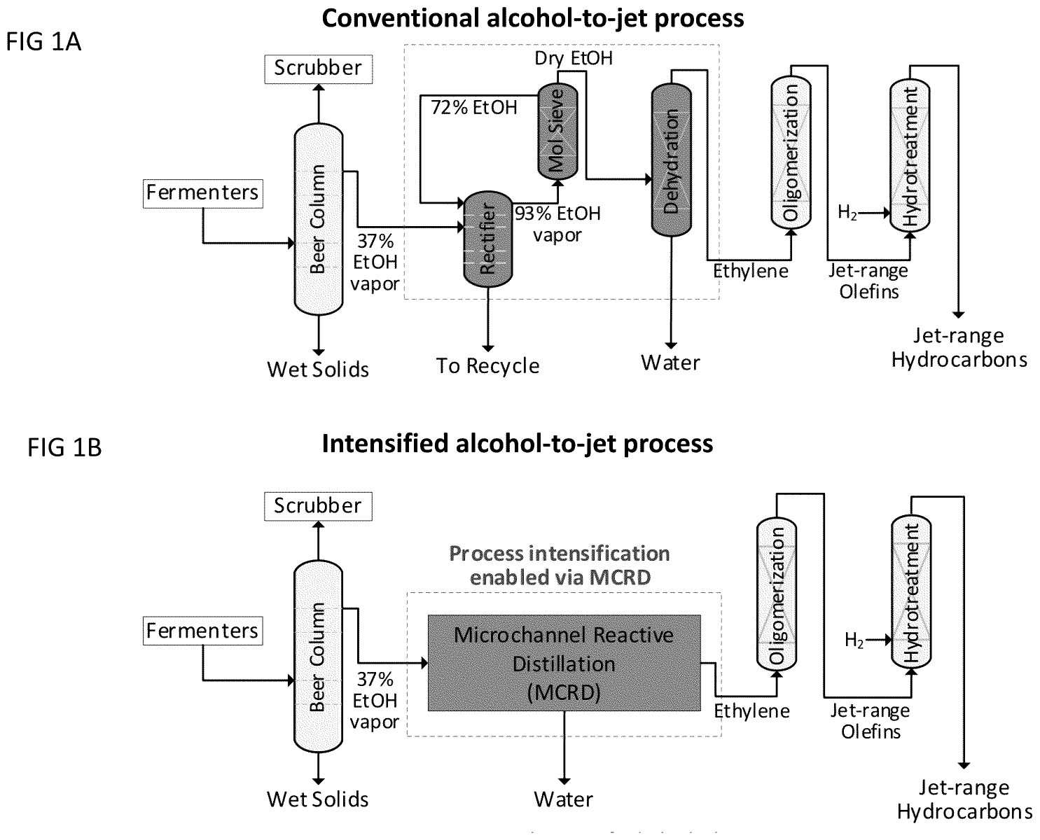

A preliminary plant-wide model of the proposed technology using Aspen modeling. The process flow diagrams for the base and proposed cases as illustrated in FIG. 1 were modeled and analyzed for total utility and capital cost. In the conventional process, the wet ethanol stream from the beer column is sent to rectifier followed by molecular sieve to produce dry ethanol. The dry ethanol is then sent to the dehydration unit to produce ethylene. In the proposed process, the wet ethanol stream is condensed, pumped to 30 atm, and then sent to a microchannel reactive distillation column packed with dehydration catalyst. The utility consumption and capital cost of the conventional process is available in the literature for ethanol production and ethanol dehydration [16]. For the proposed process, the flowsheet model was developed in Aspen Plus. Capital cost of the common equipment was estimated in Aspen Process Economic Analyzer. Capital cost for the microchannel equipment was estimated using recent data obtain internally from other PNNL staff working with the National Network for Manufacturing Innovation (NNMI) funded by DOE-AMO.

### Example II

### MCRD Device

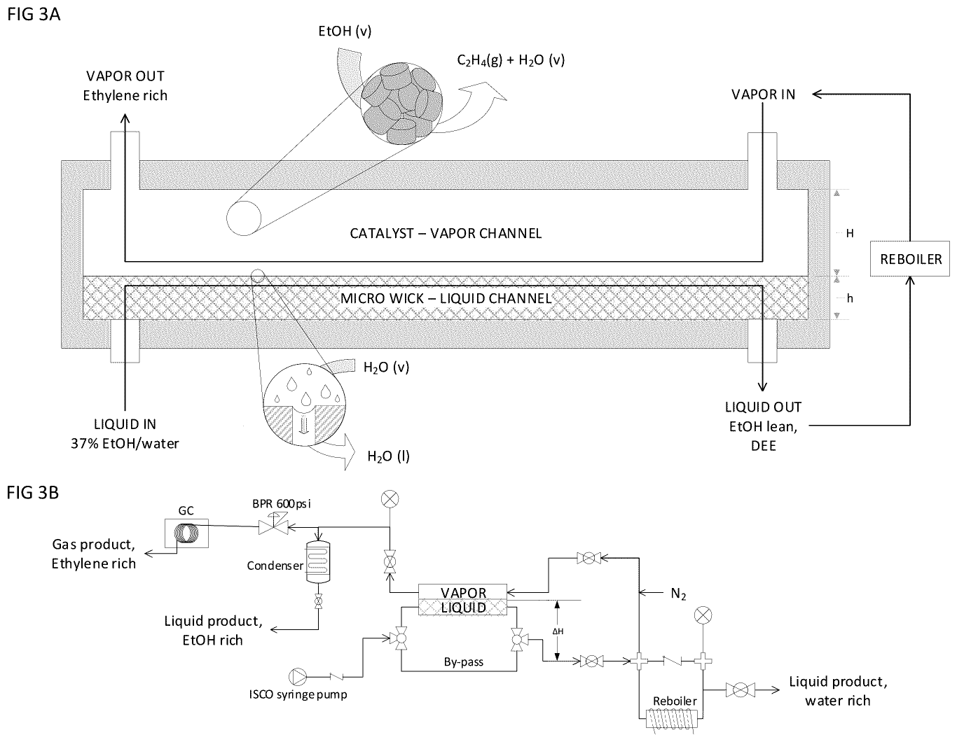

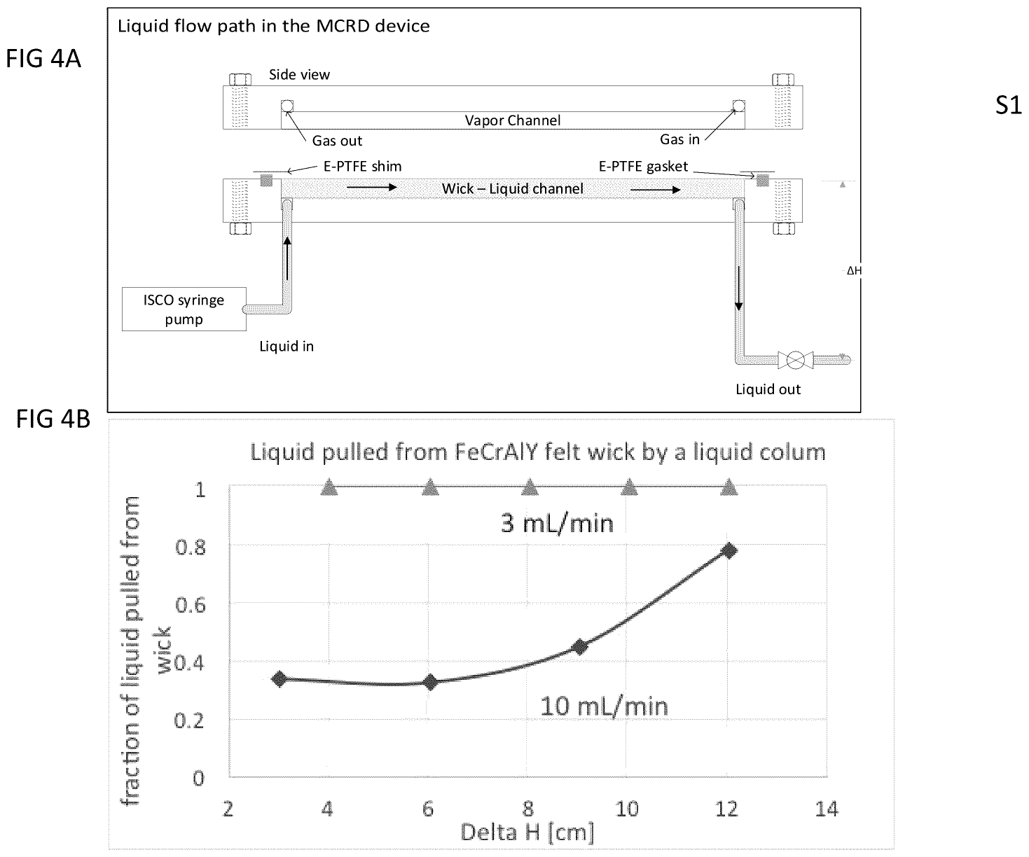

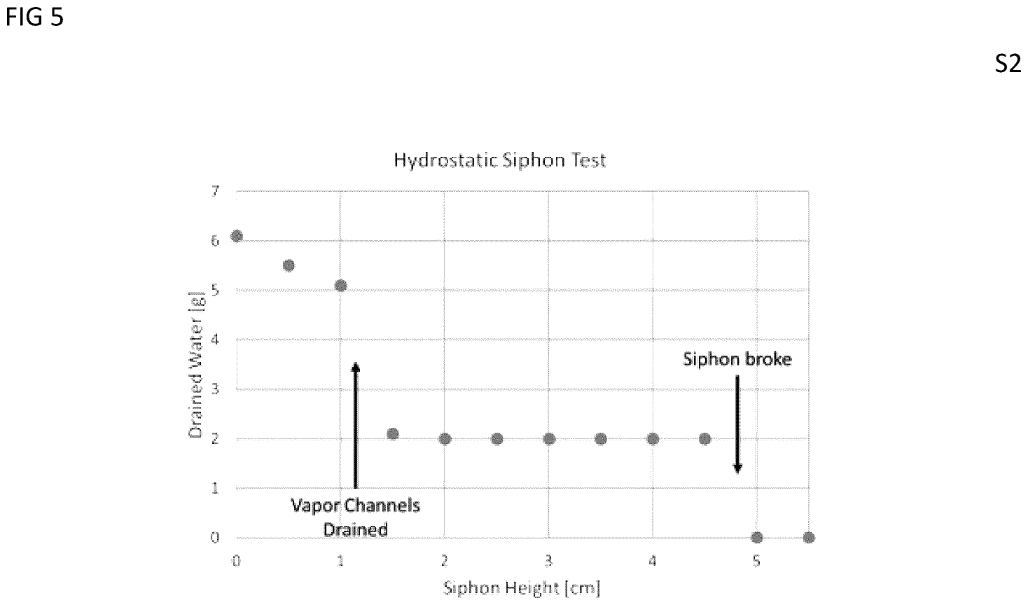

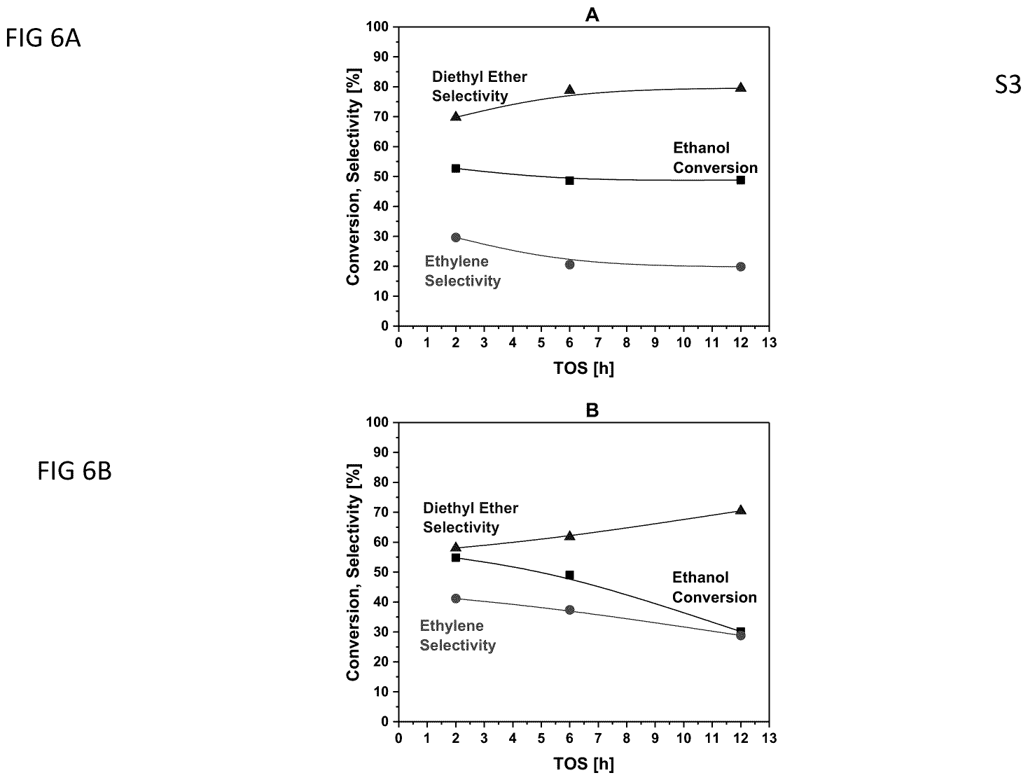

The Microchannel Reactive Distillation (MCRD) concept developed here (FIG. 3A) for the ethanol/water separation builds on our prior microchannel distillation development efforts [8, 9]. In this prior work, two important key concepts were identified pertaining to MCRD operability: 1) micro wick collects the liquid formed inside the device and transport it to the reboiler using a siphon-like flow pattern created by a column of liquid leaving the device, and 2) a reboiler operating at a higher temperature compared to the device, generates vapor that is transported back to the microchannel device. For the ethanol-water system some important knowledge was developed prior to the incorporation of catalyst and summarized in few key points:
1—FeCrAlY felt showed good affinity with ethanol-water mixtures in contrast to stainless steel wicks that showed to be effective in non-polar liquids with very low surface tension. For ethanol/water mixtures, the creation of continuous liquid flow was not possible with SS wicks. The sustained effect of the height of the column pulling liquid from the FeCrAlY wick as a function of the column height (ΔH) is presented in FIG. 4A-4B.
2—The upper limit of the wicking capacity was estimated to be at least 3 mL/min by measuring the height of the column necessary to evacuate the liquid in the wick (FIG. 5A); however, higher liquid flows can be consistently maintained (e.g. 10 mL/min) as long as enough pulling from the liquid is maintained. At a constant liquid flow (3 mL/min) the FIG. S2B shows that the liquid flow can be maintained in a range of ΔH (1.5-4.5 cm).
3—Sealing of the device was achieved with ePTFE gasket. ePTFE (expanded polytetrafluoro ethylene) is chemically resistant to steam with and extended operation temperature of 260° C. Sealing of the device using ePTFE gasket and shim is depicted in FIG. 6A-6B.

After testing the FeCrAlY wick system in continuous it was integrated in the MCRD scheme shown in FIG. 4. ΔH was chosen 3.5 cm in order to maintain a constant pull of liquid of the device over a wide range of liquid flows (<3 ml/min). the prototype consists of 3 sections: 1) MC device in which liquid that is condensed absorbs on the wick and transported the liquid outlet while the liquid inlet maintains a constant liquid delivery, 2) Reboiler that receives the liquid column from the MC device, reboiler temperature must be maintained >5° C. above the temperature of the MC device in order to enrich the vapor generated in ethanol and to maintains vapor that is going to be transported back to the MC device, and 3) condenser where the vapors from the MC device are transported using a small N₂ flow. The use of N₂ is optional if enough ethylene is formed to allow exit gas flow in order to maintain pressure in the system.

### Example III

### MCRD Separation Efficiency Demonstration

Separation tests were performed to assess how the MCRD would perform for ethanol distillation alone. These separation tests were performed at 400 psi, to ensure that liquid would be maintained in the MCRD, while liquid feed was varied from 0.01 mL/min to 0.10 mL/min. Gas flow was fixed at 4.2 SCCM to ensure no disturbance of the liquid dripping to the reboiler. In general, the expected trend of lower flow rates yielding higher separations was observed (see Table 2).

TABLE 2
Results for the hydraulic tests as a
function of flowrate and temperature.

| Feed Flow | System | Condenser | Reboiler | N_{stages} |
|---|---|---|---|---|
| Rate | Temperature | [EtOH | [EtOH | [Fenske |
| [mL/min] | [° C.] | wt %] | wt %] | Equation] |
| 0.01 | 180 | 54.6 | 13.8 | 2.30 |
| 0.1 | 180 | 55.0 | 20.2 | 1.80 |
| 0.01 | 200 | 43.8 | 10.2 | 2.20 |
| 0.05 | 200 | 35.6 | 5.8 | 2.49 |
| 0.1 | 200 | 33.4 | 16.3 | 1.08 |

At 180° C., 2.30 stages were achieved at 0.01 mL/min feed rate. Increasing the temperature to 200° C. did not seem to significantly impact the separation of the system. The highest separation of 2.49 stages was achieved at a feed rate of 0.5 mL/min at 200° C. This gives a HETP of 3.61 cm, traditional distillation tower packing for ethanol/water has a typical HETP of ˜30 cm. Thus, the MCRD is able to provide 8.3× reduction in HETP.

### Example IV

### Vapor-Liquid Equilibrium for Ethanol-Water Mixtures

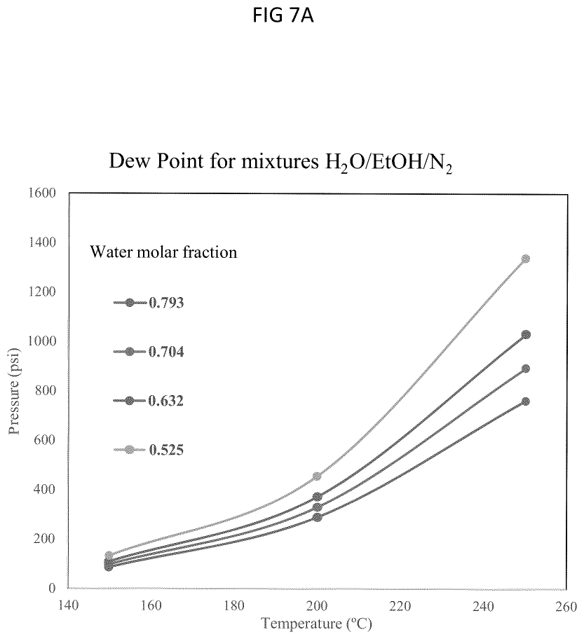

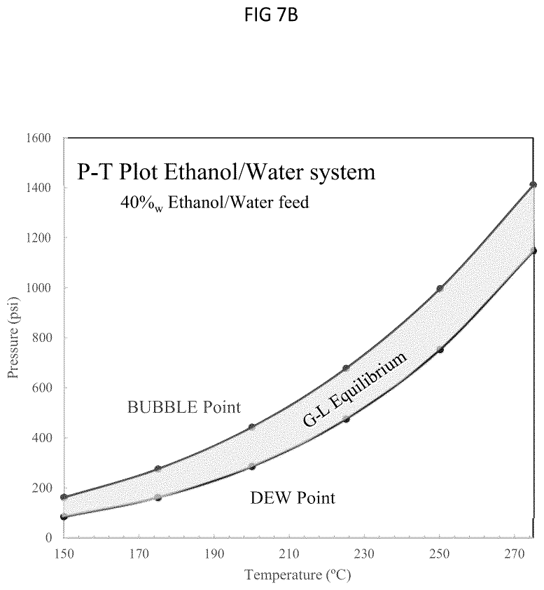

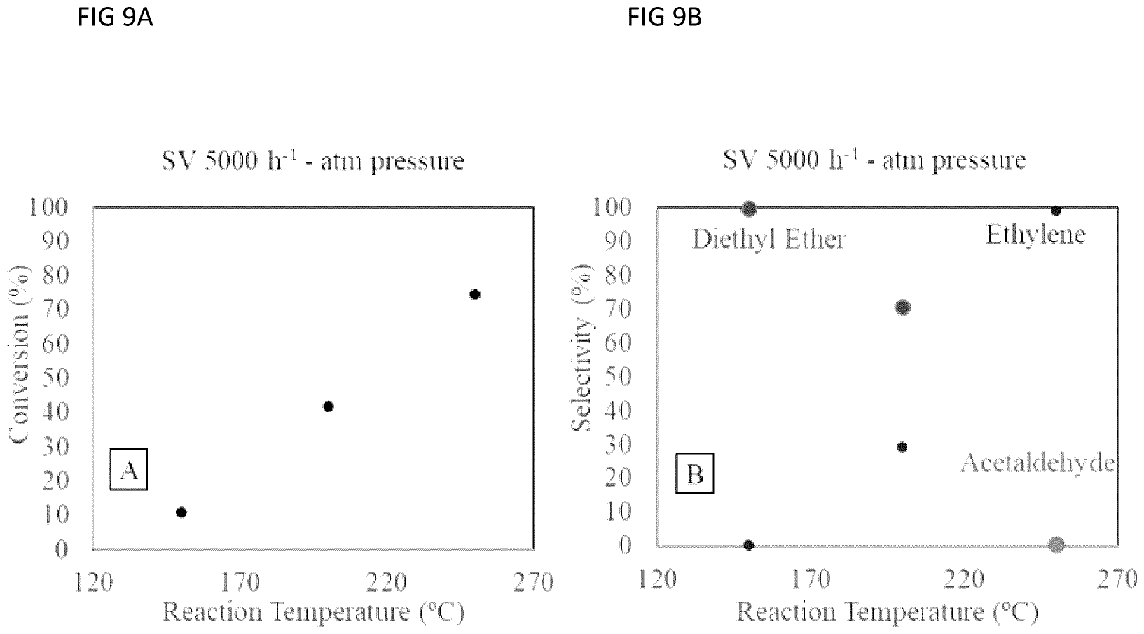

The formation of liquid drops in vapors, and vapor bubbles in liquids (dew, bubble points respectively) in the ethanol/water system is illustrated in FIG. 9. These dew and bubble values were calculated using Raoult's law with Antoine's values for partial pressure (yᵢ=1, xᵢ=1, respectively). Dew point is particularly important for the design of the catalytic RMCD device because is determines the operation pressure at a given temperature necessary to carry out the separation; as higher temperature is necessary to reach higher catalytic rates of higher selectivity, a higher pressure is necessary to obtain a liquid that is absorbed in the wick and transported to the re-boiler. FIG. 7A shows that pressure necessary to reach the dew point increases exponentially with temperature, for example a mixture 40% w ethanol (18.7/10.56/71.6%ₘₒₗ EtOH/N₂/H₂O) has a dew point of 108 psi at 150° C., this value increases to 372 and 1031 psi at 200 and 250° C., respectively. The effect of pressure in the dew point is also more evident with less water content in the mixture (1340 psi when water content decreases to 52.5% mol.

The pressure necessary to reach the dew point will determine the operation pressure of the device. This pressure in turns determines the fabrication materials for the device and pressure/temperature ratings. Higher pressure also shifts the equilibrium towards the side with fewer moles (reactants side in the ethanol dehydration case). Operation conditions (P, T) are determined by the conditions necessary to perform the separation (dew point), temperature necessary to perform the chemical conversion (175-225° C. range) that will be limited to the shaded area in FIG. 9B.

### Example V

### Reactive-Separation Thermodynamic Predictions

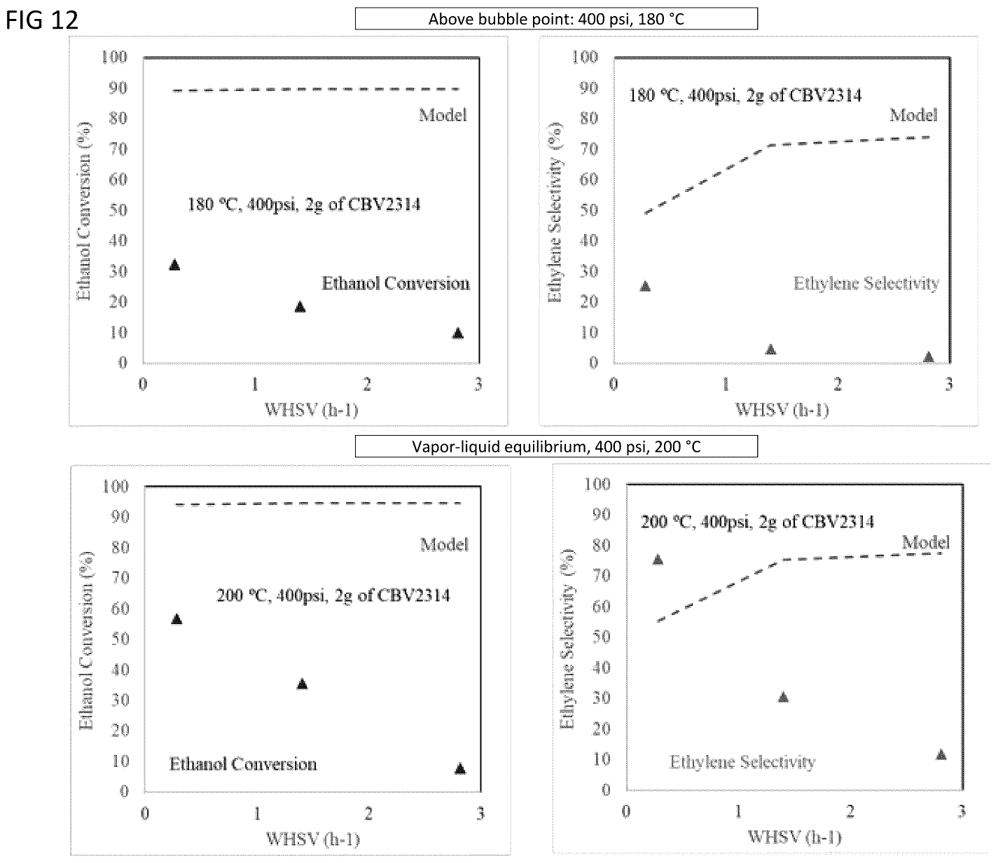

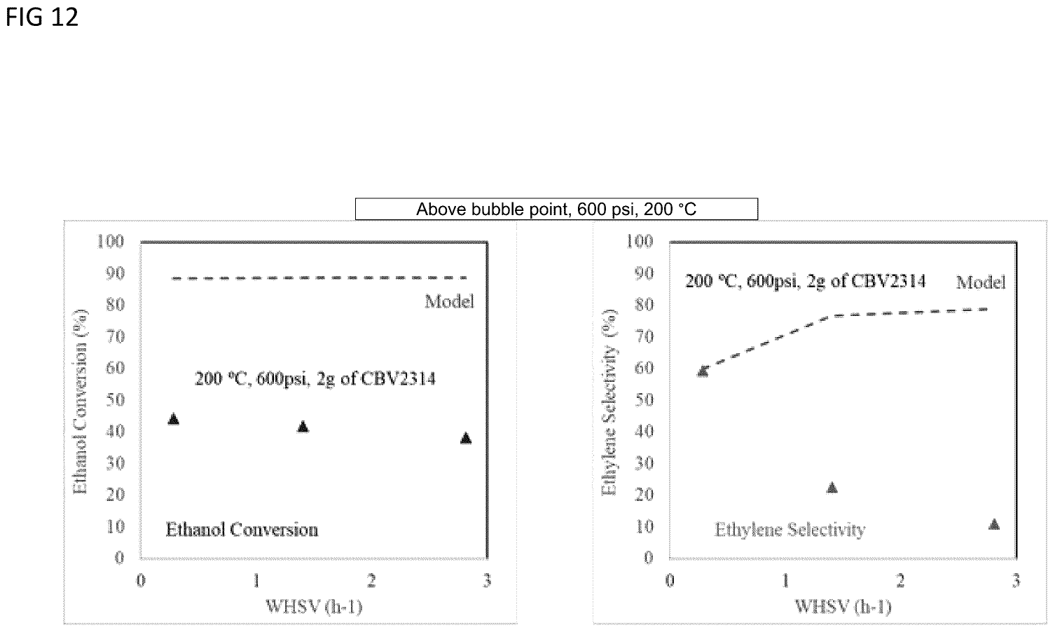

The effect of total pressure effect in ethanol conversion and the selectivity towards ethylene can be analyzed using the ChemCAD model (Gibbs reactor model) data presented in FIG. 12. At lower reaction temperatures the ethylene selectivity is lower than at higher temperatures (Dewpoint=175 psi); as the temperature is increased, pressure necessary to reach the dew point increases exponentially, and the selectivity towards ethylene is increased (88% @ 980 psi, 250° C.). Under all conditions full ethanol conversion and selectivity towards ethylene cannot be reached due to the limitation of pressure. This contrasts with the literature that shows full selectivity towards ethylene at reaction temperature as low as 220° C. at atmospheric pressure [17].

### Example VI

### Ethanol Dehydration Catalysis

A broad variety of solid acids catalyze ethanol dehydration. Although nature of acid catalysts [11, 18-23], and their physical characteristics (e.g. sites proximity, and pore confinement) have been considered as potential key factors for improved performance [17, 24, 25]; reaction conditions govern the coverage of surface species, the underlying reaction mechanism, and resulting product selectivity [24-26]. Equilibrium calculations show that, at atmospheric pressure, ethanol conversion is complete at mild reaction temperatures (100-200° C.); however, selectivity towards diethyl ether is only favored at higher temperatures (100% selectivity at T>220° C.) [17]. Water (steam) has detrimental effect in solid catalysts; particularly affecting the crystallization and structural changes in oxide supports [27], the presence of liquid water may also dissolve components (binders) of catalysts; one of the strategies to decrease the damage by steam is to operate at conditions above the dew point (vaporization of liquid water inside pores causes textural damage).

The scope of MCRD development requires to operate at conditions in the equilibrium, where liquid droplets collected in the wick are transported out of the device, and the ethanol rich vapor phase reacts with the catalyst inside the vapor channel. The residence time in the wick will favor the vaporization and at the same time avoids the formation of liquid droplets in the catalyst pores. In this study, we used two set of reaction conditions: 1) gas phase catalyst screening (225° C., 200 psi), and 2) G-L equilibrium (above the dew point) to study the performance of MCRD device. Dew point equilibrium calculations (FIG. 1A) show that the pressure necessary to create condensed liquid growths exponentially at higher temperatures.

The reaction conditions govern the coverage of key surface species which in turn has a significant role in determining the dominant reaction mechanism and product selectivity. A higher reaction temperature, higher site time and lower ethanol partial pressure favor a higher ethene selectivity. Meanwhile, the absence of a water inhibition effect makes the dehydration of aqueous bio-ethanol an attractive option for the production of bio-ethene, which can serve as a feedstock for the chemical industry. Last but not the least, the observed carbon number dependence of the standard activation entropies is in agreement with the higher reactivity of higher alcohols such as butanol compared to ethanol.

### Example VII

### Catalyst Screening

Solid acid catalysts were evaluated for ethanol dehydration activity at low temperature (150-250° C.) and elevated pressure, up to 400 psi, in order to identity a suitable formulation for use in the MCRD device. [28] Catalysts evaluated include HZSM-5 (Si/Al=23) and γ-Al₂O₃, the traditional ethanol dehydration catalyst; these results are summarized in Table 2 below. Traditional ethanol dehydration over

Al₂O₃ is typically performed using nearly dry (95% w/w) ethanol, at higher temperatures (300-500° C.), and low pressure (14-28 psi). From the conditions screening, it's clear that the higher temperature that the MCRD could be run at, the better off the reaction will be. However, to operate the MCRD, a liquid phase must be maintained, thus pressure must also increase with temperature. Were the MCRD to be operated at a typical ethanol dehydration temperature (300° C.), the pressure would need to be over 1500 psi to maintain any liquid phase. To facilitate relatively fast screening and still somewhat match conditions needed in the MCRD the conditions chosen to screen additional catalysts were: temperature 225° C., pressure 200 psi, and WHSV 3.22h⁻¹. The lower temperature, combined with higher pressure, and additional water caused γ-Al₂O₃, a traditional industrial catalyst for ethanol dehydration, to be almost entirely inactive (<2% conversion of ethanol). Other catalysts that typically perform well that were undermined by the reaction conditions were: both Zr—SiO₂ and AgW—Al₂O₃, ethanol conversions <2% and 5% respectively. While the data in the table is reported at 5,000 h⁻¹, these catalysts showed negligible activity at GHSV as low as 1,200 h⁻¹. Only the zeolites (H-ZSM-5 (Si/Al=23) and permeations of that) showed any reasonable activity (conversions >10%) under the conditions studied. Because of this, H-ZSM-5 (Si/Al=23) was selected for further screenings and permutations.

To test the role of acid sites, several permutations of the H-ZSM-5 were tested. A similar H-ZSM-5 (Si/Al=80) was tested to see lower acidity, both in strength and total number of sites. The result was a slight decrease in ethanol conversion (44.0% compared to 48.6% over Si/Al=23) and a marked decrease in the ethylene selectivity (10.2% compared to 20.5% over Si/Al=23). To selectivity mask only the stronger acid sites, P (3 wt. %) was doped onto the H-ZSM-5 (Si/Al=23). The results were similar to the H-ZSM-5 (Si/Al=80) with a slight decrease in ethanol conversion (45.9%) and a marked decrease in ethylene selectivity (12.2%). Testing the opposite trend, additional acid sites in the form of phosphotungstic acid (PWA) and trifluoromethanesulfonic acid (Triflic) were doped onto the H-ZSM-5 (Si/Al=23). The PWA increased both conversion and ethylene selectivity slightly, 49.3% and 22.0% respectively. While Triflic had a negligible effect on ethanol conversion (47.9%) but significantly increase the ethylene selectivity, 35.6%. This suggests that at these lower temperatures, not just more acidic sites, but stronger acidic sites are needed to support ethanol dehydration.

TABLE 3
Activity summary of ethanol dehydration over screened
catalysts at 225° C., 200 psi, WHSV 3.22h−1,
feedstock 40 wt. % ethanol in water.

| | | Ethylene | |
|---|---|---|---|
| | Conversion | Selectivity | Ethylene yield |
| | (%) | (C %) | (% mass gE/gEtOH) |
| Triflic-H-ZSM-5(23) | 47.9 | 35.6 | 8.48 |
| PWA- H-ZSM-5(23) | 49.29 | 22.05 | 6.72 |
| H-ZSM-5(23) | 48.6 | 20.53 | 5.74 |
| H-ZSM-5(80) | 44.01 | 10.25 | 3.56 |
| P-doped H-ZSM-5(23) | 45.9 | 12.25 | 3.14 |
| AgW—Al₂O₃ | 5 | 2 | 0 |
| Zr₃—SiO₂ | <2 | 5 | 0.002 |
| Zr₂₀—SiO₂ | <2 | 6 | 0.006 |
| γ-Al₂O₃ | <2 | 20 | 0.23 |

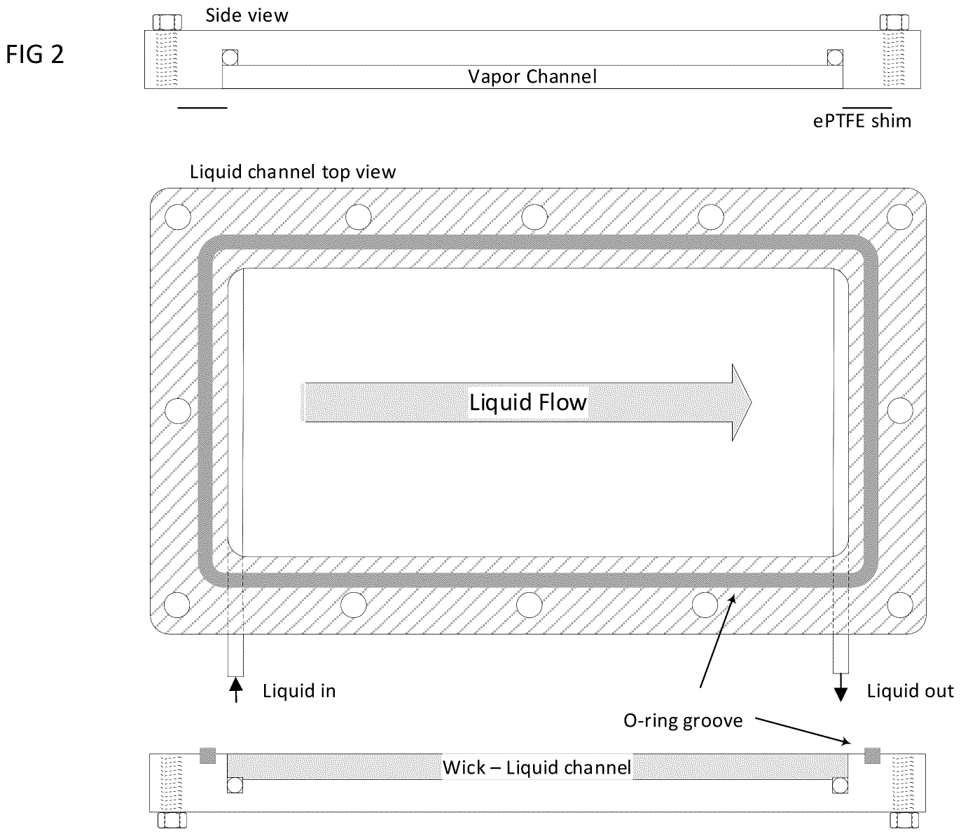

Based on the catalyst screening, Triflic promoted H-ZSM-5 (Si/Al=23) appeared to be the best candidate to test in the MCRD, however, catalyst stability was still unknown. To test catalyst stability H-ZSM-5 (Si/Al=23), both with and without Triflic promotion, was tested for 12 h time on stream (TOS), FIG. 2. Initial activity for both matched with the initial screening data, with Triflic promotion enhancing both ethanol conversion and ethylene selectivity. The unmodified H-ZSM-5 (Si/Al=23) then continued to show roughly the same activity, ethanol conversion decreased slightly but remained relatively stable at 48.8% until 12 h TOS. The ethylene selectivity behaved similarly, decreasing slightly but remaining relatively steady at 19.8% until 12 h TOS. In contrast, the Triflic promoted H-ZSM-5 (Si/Al=23) showed loss of both ethanol conversion and ethylene selectivity. While the initial ethanol conversion and ethylene selectivity were high, 54.8% and 41.1% respectively, by 12 h TOS both had dropped, ethanol conversion 30.1% and ethylene selectivity 28.8%. Operation of the MCRD requires operating for days on end, thus catalyst stability is a key factor. Based on this, despite the Triflic promotion clearly enhancing the initial catalyst activity, the loss of activity made it ill-suited for the MCRD and the H-ZSM-5 (Si/Al=23) was selected for operations.

### Example VII

### Parametric Evaluation

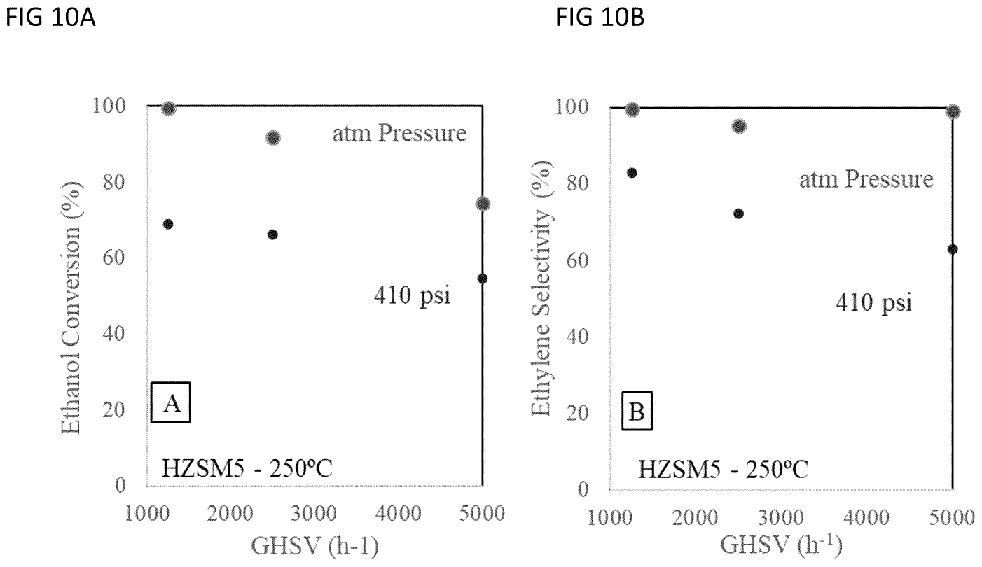

H-ZSM-5 (Si/Al=23) was selected for further evaluation of process parameters including temperature, pressure and residence time (WHSV) were evaluated, data are shown in FIGS. 9 and 10. Catalytic studies in literature are focused on finding catalysts and conditions with three main performance characteristics: high ethanol conversion, high ethylene selectivity, and high catalyst stability. FIG. 9A shows that conversion increases linearly with reaction temperature [10]. FIG. 9B indicates that small increments in temperature cause a big increase in ethylene selectivity, ethylene selectivity is complete at 250° C. (literature shows the same trend, selectivity towards ethylene is only complete after a small temperature window at about 225-250° C.), similarly to literature results that indicate that the formation of ethylene is intermediated by the formation of DEE, and that DEE decomposition (by hydrolysis) is rate limiting. At 250° C., dehydrogenation reaction is activated, and traces of acetaldehyde are formed.

Operation at higher total pressure, as predicted by ChemCAD modelling, decreases conversion and ethylene selectivity compared to atmospheric pressure operation as shown in FIG. 10. At 250° C., ethanol conversion drops when reactor pressure is increased to 410 psi, FIG. 10A shows that space velocity has no significant effect in ethanol conversion. Ethylene selectivity (FIG. 10B) is also affected by the increased pressure, while selectivity towards ethylene is full atmospheric pressure, it decreases to the range 60-80% at 410 psi.

### Example VIII

### Catalyst Stability

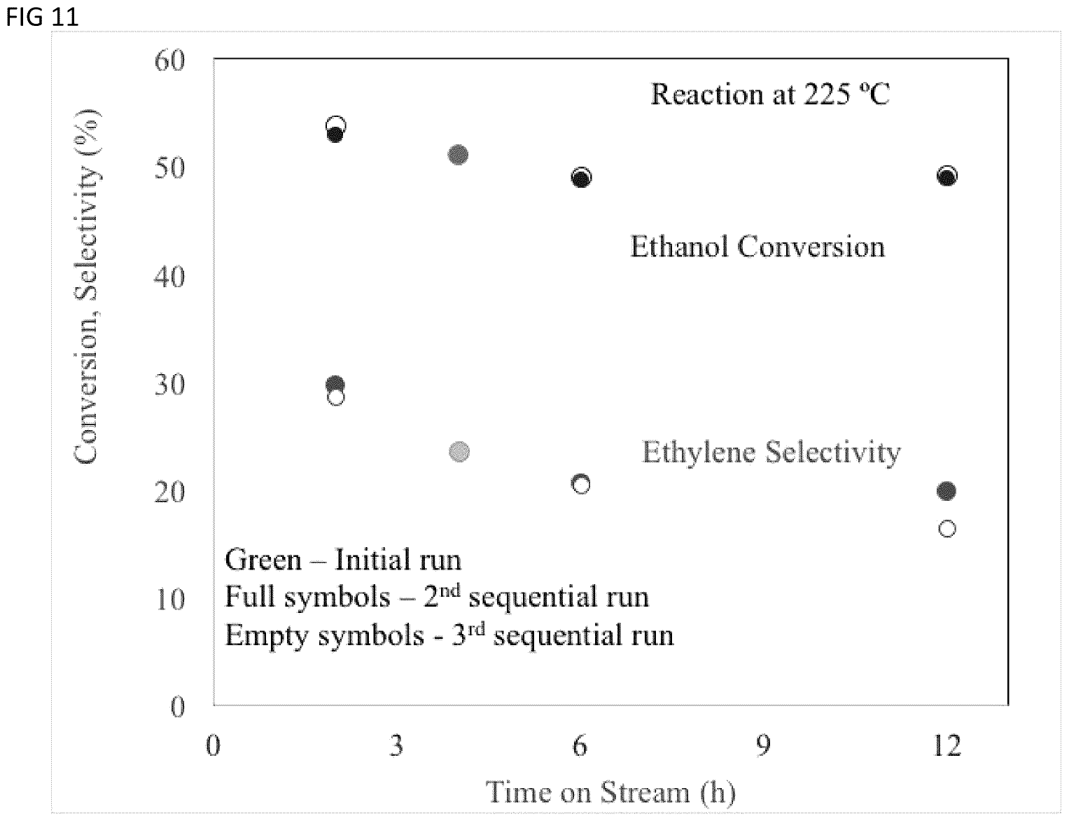

Catalysts used in ethanol dehydration show deactivation over time, but the analysis of performance over long periods of time is not often reported, coke formation is the main reason presented for catalyst deactivation [18, 19]. In order to validate the use of ZSM5 catalyst for the demonstration of RMCD device, we performed three sequential runs over the same catalyst sample, results are presented in FIG. 11. After loading the fresh catalyst, the reaction was allowed to proceed for 4 hours, then the reactor was maintained under N₂ flow overnight, the reaction was resumed the next day and the reaction was allowed to proceed for 12 hours; the next day, a third cycle was run after flushing the catalyst overnight under flowing N₂.

FIG. 11 shows that over the course of the 12 h reaction, ethylene selectivity drops drastically (28 to 16%) while the ethanol selectivity was less affected (53.7 to 48.8%). This fast deactivation seems to be happening the first hours of catalysis. After flushing the catalyst overnight, under N₂ flow, the activity of the fresh catalyst is recovered. This recovery of activity indicates that no coke is formed, and that on the contrary, strongly adsorbed species (ethoxy) may be adsorbing and accumulating during catalysis causing deactivation as the reaction proceeds. Some important observations of this catalyst stability test can be pointed out: 1) a fast drop in activity occurs during the first few hours of reaction, 2) the deactivation slows down as the reaction proceeds, 3) the loss in activity is reversible after flushing the catalyst with nitrogen overnight, and 4) at 225° C. no dehydrogenation or coke formation activity was only traces of acetaldehyde were detected by GCMS (<0.5% c), and the activity was recovered after 3 reaction cycles.

### Example IX

### RMCD Catalytic Tests

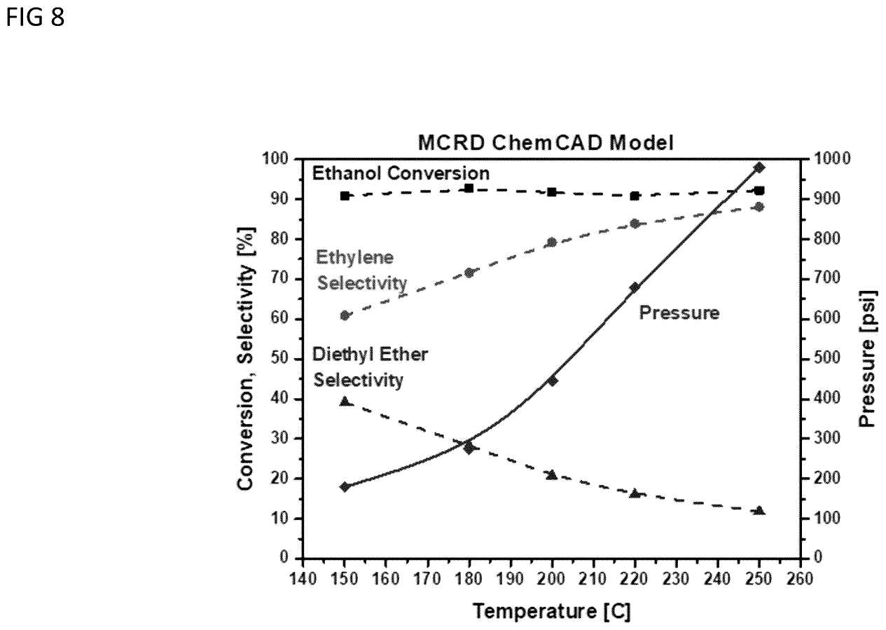

The development of the RMCD concept involves the study of operation variables that in the case of ethanol dehydration faces some critical challenges compared to the separate operations ethanol-water distillation and catalytic dehydration. First, in the case of ethanol-water distillation, RMCD main challenge consists on providing operation conditions under a vapor-liquid equilibrium that allows the formation of a liquid phase (operation above dew point) that is transported to the reboiler and enriched in ethanol as it is returned to the device. This is only provided by an increased operation pressure in contrast to regular (packed/bed, plates) distillation (FIG. 7). In the case of catalytic dehydration high temperature, that provides high activity (ethanol conversion rates) and high selectivity (ethylene selectivity) are necessary; however, pressure necessary to create the liquid phase affecting the chemical equilibrium (Le Chatelier), favoring the hydrated products (FIG. 8). Catalyst formulation does not offer the possibility to provide significant improvements (full ethanol conversion or ethylene selectivity) given equilibrium constraints (FIG. 12). Water separation from the vapor favors the equilibrium towards dehydration products (ethylene and DEE). The main goal from this section is to show how the integration of distillation and catalytic dehydration affects the chemical equilibrium is affected using the RMCD device.

In order to evaluate the catalyst performance when incorporated to the RMCD device, 2.0 g of ZSM5-CBV2314 catalyst (60-80 mesh) was loaded inside the vapor channel of the RMCD device and pressurized using N₂ flow (100 sccm). The liquid path was created flooding the wick and liquid channel with the feed (40% w ethanol/water mix), the siphon that pulls liquid from the device is created with a liquid column connected to the outlet. After this flow system is created, and all the liquid product is collected in the reboiler outlet (no liquid collected in the condenser is verified for 12 hours), the heating starts (at a rate of 5° C./min). After the temperature is reached, the system is allowed to run for 2 h, after this time, liquids are purged from reboiler outlet and condenser. This marks the time zero for all the conditions presented in this section. Each set of reaction conditions was allowed to run after 10 mL of feedstock was processed (16.6, 3.3, 1.7 h for 0.01, 0.05, and 0.1 mL/min feed flow), the liquids (from rebuild and condenser) are collected and analyzed by LC.

FIG. 12 shows key catalyst performance data (ethanol conversion and ethylene selectivity) for the ethanol dehydration using the RMCD device and compared to the ChemCAD model for three different reaction conditions: A) above the bubble point, 400 psi, 180° C., B) vapor-liquid equilibrium, 400 psi, 200° C., and C) above the bubble point, 600 psi, 200° C. Under these conditions, liquid is formed in the device and a direct comparison to the conditions used for the catalyst screening and parametric study is not possible (these were performed in the gas phase). Model estimations show high ethanol conversion at the three conditions selected (>90%). For the same model calculations selectivity increases as the space velocity increases. This seems to be due to the fact that the equilibrium is shifted toward hydration side for short contact times (reverse ethylene hydration is favored or recombination of ethylene with ethoxy intermediates to form DEE).

Experimental results presented in FIG. 12 show that both ethanol conversion (blue) and ethylene selectivity (green) increase at lower space velocities (higher contact times). The incomplete ethanol and low selectivity towards ethylene may be explained from three points of view: 1) chemical equilibrium is shifted towards ethanol by ethylene hydration, 2) as the reaction proceeds, ethylene product reacts with adsorbed ethoxy intermediates to produce diethyl ether, and 3) ethanol dehydration mechanism involves a bimolecular pathway to generate diethyl ether, which is more energetically favored than the formation of ethanol monomers [29]. Regardless of the mechanism, it is interesting to note that selectivity shifts towards DEE as the space velocity is increased, this indicates that ethylene formation may be kinetically limited. Table 4 shows a summary of the catalytic results (ethanol conversion and ethylene selectivity) combined with the separation obtained for a set of conditions above the dew point. These results can be summarized in a few key points:
1—Separation (#of stages) decrease as the inlet flow is increased, this indicates that the characteristic length of the wick (9 mm), and the resulting contact time, is the limiting variable in the separation process.
2—Catalytic performance (ethanol conversion and ethylene selectivity) decrease as space velocity is increased, this indicates that on the one hand the load of catalyst is the limiting factor in the dehydration reaction, and that increase contact time shifts the equilibrium towards the formation of DEE which can be determined as the rate limiting step in the ethylene formation.
3—The approach to equilibrium and results obtained by the ChemCAD model are only favored at the lowest space velocities (higher contact times) for a single pass in which nitrogen is continuously removing vapors from the vapor channel.

TABLE 4
Summary of catalytic results (ethanol conversion and ethylene selectivity) and separation
stages obtained with the RMCD device. Reaction conditions: Nitrogen flow: 5 sccm,
2 g of CBV2314-HZSM5 catalyst. Pressure is created and maintained using Nitrogen
flow, space velocity is changed by increments of the liquid flow.

| Temperature | Pressure | Liquid Feed | WHSV | EtOH Conversion | Selectivity | Separation |
|---|---|---|---|---|---|---|
| (° C.) | (psi) | Rate [mL/min] | [h⁻¹] | [%] | [%_{C} Ethylene] | [# Stages] |
| 180 | 400 | 0.01 | 0.28 | 32.50 | 25.44 | 2.08 |
| | | 0.05 | 1.40 | 18.65 | 4.71 | 1.23 |
| | | 0.10 | 2.81 | 9.92 | 2.32 | 1.16 |
| 200 | 600 | 0.01 | 0.28 | 44.40 | 59.44 | 2.63 |
| | | 0.05 | 1.40 | 41.84 | 22.47 | 1.90 |
| | | 0.10 | 2.81 | 38.20 | 10.95 | 2.26 |
| | 400 | 0.01 | 0.28 | 56.79 | 75.75 | 1.68 |
| | | 0.05 | 1.40 | 35.74 | 30.59 | 2.07 |
| | | 0.10 | 2.81 | 7.82 | 11.91 | 1.86 |
| 220 | 600 | 0.01 | 0.28 | 70.71 | 90.72 | 2.65 |
| | | 0.05 | 1.40 | 41.39 | 83.61 | 1.26 |
| | | 0.10 | 2.81 | 43.68 | 45.57 | 1.40 |
| | 500 | 0.01 | 0.28 | 78.27 | 91.45 | 1.45 |
| | | 0.05 | 1.40 | 40.90 | 54.06 | 1.18 |
| | | 0.10 | 2.81 | 44.89 | 39.19 | 0.76 |
| | 400 | 0.01 | 0.28 | 32.41 | 48.76 | 0^{§} |
| | | 0.05 | 1.40 | 22.54 | 23.14 | 0 |
| | | 0.10 | 2.81 | 9.79 | 16.66 | 0 |

^{§}these conditions fall under the dew point; no liquid is formed in the device.

Shown in in Table 5 are the best results achieved to-date. When operating at 220° C., 600 psig, and 0.28 hr⁻¹ ethanol conversion of 71% and ethylene selectivity of 91% was obtained, with the current MCRD configuration. Selectivity to ethylene was far greater than that predicted for the reaction-only model (52%) and was similar to that as predicted by the model (84%). We note that experimental error particularly at the low flow rates (0.28 hr⁻¹) account for the higher ethylene selectivity as predicted by ChemCAD. However, the conversion as demonstrated by experiment (71%) is lower than that predicted the model (91%). Future MCRD improvements are expected to close this gap. Additionally, 2.7 theoretical stages of separation were achieved. This gives a HETP of 3.3 cm, where traditional distillation tower packing for ethanol/water has a typical HETP of ˜30 cm. Thus, the MCRD is able to provide 9× reduction in HETP.

TABLE 5
Comparison of reaction only (Gibbs reactor) and reaction
and separation (RD), as predicted by ChemCAD modeling,
and actual experimental data for ethanol dehydration at
220° C., 600 psi, and 0.28 hr⁻¹.

| | Reaction | Reaction and | Reaction and |
|---|---|---|---|
| | Only - | Separation - | Separation - |
| System | Model | Model | Experimental |
| Ethanol Conversion [%] | 80 | 91 | 71 |
| Ethylene Selectivity [%] | 52 | 84 | 91 |
| Separation [# Stages] | N/A | ~5* | 2.7 |

*This is estimated from data obtained from similar device, optimized for propane/propylene separations

These results are encouraging the support the proof of concept demonstration for microchannel reactive distillation. However, improvements are required in order to obtain the model predicted results. Future improvements to the device itself and through recycle of undesired DEE byproduct is expected to produce improved improvements. Overcoming the kinetic limitations of the device require the use of either a wick with a higher characteristic length (L=9 cm) for improved separation (#of stages) and the use of a higher load of catalyst (increased contact time). Approximately 5 stages of separation is required for this reaction. The use of nitrogen gas was also used for pressurizing the device and as internal standard for concentration/flows calculation. This is useful for transporting the vapors from the reboiler back to the device and to work as internal standard for demonstration purposes; however, it causes the device to work on a single pass basis, vapors in the vapor channel are continuously removed from the device. In an ideal situation with no carrier gas, ethylene is the only product leaving the device allowing for a continuous recirculation. Finally, recycle of produced DEE would further convert to ethylene.

### Example X

### Techno Economic Analysis

To assess the feasibility of the proposed technology, we have conducted a preliminary plant-wide model using Aspen modeling. The process flow diagrams for the base and proposed cases as illustrated in FIG. 1 were modeled and analyzed for total utility and capital cost. Key economic measures are summarized in Table 6. This preliminary analysis shows the potential for utility (energy) reduction of 35% or more by integrating the separation and reaction process together. In addition, the expensive molecular sieve to break the water/ethanol azeotrope is no longer needed. While conventional equipment was utilized in the model (being available in ASPEN) the use of optimized microchannel reactive distillation has the potential for further reduction in utility consumption due to its superior HETP. HETP will have significant effect on reflux ratio, and therefore the energy consumption related to separations. The proposed process also has the potential to significantly reduce capital cost of distillation and reactors, which may lead to a 55% total capital cost reduction.

TABLE 6
Key economic measures for the wet ethanol to ethylene portion of the base case
and proposed process flowsheets illustrated in FIG. 1 (2016 US dollar).

| | Conventional | | | | | Potential |
|---|---|---|---|---|---|---|
| | | Mol | | | Proposed | Savings |
| Utility consumption | Rectifier | Sieve | Dehydration | Total | Total | (%) |
| Natural gas | | | 709 | 709 | | |
| (BTU/lb C2=) | | | | | | |
| Cooling water | 19.5 | 6.5 | 6.08 | 32.1 | 26.8 | |
| (gal/lb C2=) | | | | | | |
| Steam (lb/lb C2=) | 0.87 | 0.04 | 1.73 | 2.64 | 2.17 | |
| Power (kWh/lb C2=) | 0.009 | 0.005 | 0.057 | 0.07 | 0.003 | |
| Utility cost (¢/lb C2=) | | | | 3.71 | 2.43 | 35% |
| Fixed Capital | 12.06 | 16.90 | 91.13 | 120.1 | 53.8 | 55% |
| Investment⁽¹⁾ (MM$) | | | | | | |

⁽¹⁾Plant capacity = 21691 kg/hr ethanol (~2000 BPD jet fuel); Balance of plant is included with similar ratio reported by Humbird et al. [15].

[1] A. A. Kiss, J.-P. Lange, B. Schuur, D. W. F. Brilman, A. G. J. van der Ham, S. R. A. Kersten, Separation technology-Making a difference in biorefineries, Biomass and Bioenergy, 95 (2016) 296-309.
[2] T. Lassmann, P. Kravanja, A. Friedl, Simulation of the downstream processing in the ethanol production from lignocellulosic biomass with ASPEN Plus® and IPSEpro, Energy, Sustainability and Society, 4 (2014) 27.
[3] J. J. Lerou, A. L. Tonkovich, L. Silva, S. Perry, J. McDaniel, Microchannel reactor architecture enables greener processes, Chemical Engineering Science, 65 (2010) 380-385.
[4] Y. Wang, Y. H. Chin, R. T. Rozmiarek, B. R. Johnson, Y. Gao, J. Watson, A. Y. L. Tonkovich, D. P. Vander Wiel, Highly active and stable Rh/MgOAl2O3 catalysts for methane steam reforming, Catalysis Today, 98 (2004) 575-581.
[5] R. Zheng, R. Diver, D. Caldwell, B. Fritz, R. Cameron, P. Humble, W. TeGrotenhuis, R. Dagle, R. Wegeng, Integrated Solar Thermochemical Reaction System for Steam Methane Reforming, Energy Procedia, 69 (2015) 1192-1200.
[6] C. Cao, J. Hu, S. Li, W. Wilcox, Y. Wang.
[7] F. Zheng, V. S. Stenkamp, W. E. TeGrotenhuis, X. Huang, D. L. King, Microchannel Distillation of JP-8 Jet Fuel for Sulfur Content Reduction, AIChE, New York, NY, United States (US).; Pacific Northwest National Lab. (PNNL), Richland, WA (United States) 2006.
[8] D. Bottenus, D. Caldwell, C. Fischer, P. Humble, M. Powell, R. Lucke, W. TeGrotenhuis, Process intensification of distillation using a microwick technology to demonstrate separation of propane and propylene, AIChE Journal, 64 (2018) 3690-3699.
[9] W. E. TeGrotenhuis, D. R. Bottenus, E. W. Hoppe, P. H. Humble, R. Lucke, M. R. Powell, Isotope Enrichment Using Microchannel Distillation Technology (Final Report); Pacific Northwest National Lab. (PNNL), Richland, WA (United States), 2018, pp. Medium: ED; Size: 76 p.
[10] G. Garbarino, R. Prasath Parameswari Vijayakumar, P. Riani, E. Finocchio, G. Busca, Ethanol and diethyl ether catalytic conversion over commercial alumina and lanthanum-doped alumina: Reaction paths, catalyst structure and coking, Applied Catalysis B: Environmental, 236 (2018) 490-500.
[11] T. K. Phung, G. Busca, Ethanol dehydration on silica-aluminas: Active sites and ethylene/diethyl ether selectivities, Catalysis Communications, 68 (2015) 110-115.
[12] Z. Song, A. Takahashi, I. Nakamura, T. Fujitani, Phosphorus-modified ZSM-5 for conversion of ethanol to propylene, Applied Catalysis A: General, 384 (2010) 201-205.
[13] R. Le Van Mao, T. M. Nguyen, G. P. Mclaughlin, The bioethanol-to-ethylene (B. E. T. E.) process, Applied Catalysis, 48 (1989) 265-277.
[14] C. L. Marchena, R. A. Frenzel, S. Gomez, L. B. Pierella, L. R. Pizzio, Tungstophosphoric acid immobilized on ammonium Y and ZSM5 zeolites: Synthesis, characterization and catalytic evaluation, Applied Catalysis B: Environmental, 130-131 (2013) 187-196.
[15] D. Humbird, R. Davis, L. Tao, C. Kinchin, D. Hsu, A. Aden, P. Schoen, J. Lukas, B. Olthof, M. Worley, D. Sexton, D. Dudgeon, Process Design and Economics for Biochemical Conversion of Lignocellulosic Biomass to Ethanol: Dilute-Acid Pretreatment and Enzymatic Hydrolysis of Corn Stover. NREL/TP-5100-47764, in: N.R.E. Laboratory (Ed.) Golden, Colorado, 2011.
[16] Ethylene from ethanol by adiabatic fixed-bed catalytic dehydration., PEP Yearbook, 2014.
[17] C.-Y. Wu, H.-S. Wu, Ethylene Formation from Ethanol Dehydration Using ZSM-5 Catalyst, ACS Omega, 2 (2017) 4287-4296.
[18] T. K. Phung, L. Proietti Hernandez, A. Lagazzo, G. Busca, Dehydration of ethanol over zeolites, silica alumina and alumina: Lewis acidity, Brønsted acidity and confinement effects, Applied Catalysis A: General, 493 (2015) 77-89.
[19] I. Takahara, M. Saito, M. Inaba, K. Murata, Dehydration of Ethanol into Ethylene over Solid Acid Catalysts, Catalysis Letters, 105 (2005) 249-252.
[20] D. Varisli, T. Dogu, G. Dogu, Ethylene and diethyl-ether production by dehydration reaction of ethanol over different heteropolyacid catalysts, Chemical Engineering Science, 62 (2007) 5349-5352.
[21] K. Ramesh, L. M. Hui, Y.-F. Han, A. Borgna, Structure and reactivity of phosphorous modified H-ZSM-5 catalysts for ethanol dehydration, Catalysis Communications, 10 (2009) 567-571.
[22] H. Xin, X. Li, Y. Fang, X. Yi, W. Hu, Y. Chu, F. Zhang, A. Zheng, H. Zhang, X. Li, Catalytic dehydration of ethanol over post-treated ZSM-5 zeolites, Journal of Catalysis, 312 (2014) 204-215.
[23] D. Fan, D. J. Dai, H. S. Wu, Ethylene Formation by Catalytic Dehydration of Ethanol with Industrial Considerations, Materials (Basel), 6 (2012) 101-115.
[24] K. Golabek, E. Tabor, V. Pashkova, J. Dedecek, K. Tarach, K. Góra-Marek, The proximity of aluminium atoms influences the reaction pathway of ethanol transformation over zeolite ZSM-5, Communications Chemistry, 3 (2020) 25.
[25] J. F. DeWilde, H. Chiang, D. A. Hickman, C. R. Ho, A. Bhan, Kinetics and Mechanism of Ethanol Dehydration on γ-Al2O3: The Critical Role of Dimer Inhibition, ACS Catalysis, 3 (2013) 798-807.
[26] H. Arai, J.-I. Take, Y. Saito, Y. Yoneda, Ethanol dehydration on alumina catalysts: I. The thermal desorption of surface compounds, Journal of Catalysis, 9 (1967) 146-153.
[27] P. Forzatti, L. Lietti, Catalyst deactivation, Catalysis Today, 52 (1999) 165-181.
[28] M. Zhang, Y. Yu, Dehydration of Ethanol to Ethylene, Industrial & Engineering Chemistry Research, 52 (2013) 9505-9514.
[29] H. Chiang, A. Bhan, Catalytic consequences of hydroxyl group location on the rate and mechanism of parallel dehydration reactions of ethanol over acidic zeolites, Journal of Catalysis, 271 (2010) 251-261.

1. Apparatus for the combined separation and conversion of an oxygenate in aqueous solution to more volatile intermediates for gas phase conversion, comprising:
a liquid inlet connected to a wicking microchannel;
an outlet connected to the wicking microchannel;
a vapor microchannel adjacent the wicking microchannel;
a vapor inlet connected to the vapor microchannel and a vapor outlet connected to the vapor microchannel;
a hydrophilic wick disposed in the wicking microchannel; and
a hydrophobic solid acid catalyst disposed in the vapor microchannel.

2. The apparatus of claim 1 wherein the hydrophilic wick comprises a hydrophilic FeCrAlY foam or felt.

3-4. (canceled)

5. A method of combined separation and conversion of an oxygenate in aqueous solution to more volatile intermediates for gas phase conversion, comprising;
passing a liquid comprising oxygenates into a reaction chamber;
wherein the reaction chamber comprises a wicking microchannel comprising a hydrophilic porous wick for liquid transport, and a vapor microchannel adjacent the wick for gas phase conversion having a thickness of 1 cm or less;
wherein the hydrophilic wick comprises a hydrophilic FeCrAlY foam or felt;
wherein the vapor microchannel comprises a heterogenous catalyst;
heating the liquid in the wicking microchannel wherein oxygenate is stripped from the liquid phase and dehydrated over a hydrophobic solid acid catalyst to produce water and a gaseous dehydrated product, and wherein the produced water preferentially passes back into the aqueous solution in the wick;
wherein the aqueous solution passes through the wick and out of the reaction chamber via a first liquid outlet; and
wherein the gaseous dehydrated product passes through the vapor microchannel and out of the reaction chamber through a vapor outlet.

6. A method of combined separation and conversion of an oxygenate in aqueous solution to more volatile intermediates for gas phase conversion, comprising;
passing a liquid comprising between 1 and 80 wt. % oxygenates and 20 to 99 wt. % water into a reaction chamber;
wherein the reaction chamber comprises a wicking microchannel comprising a hydrophilic porous wick for liquid transport, and a vapor microchannel adjacent the wick for gas phase conversion having a thickness of 1 cm or less;
wherein the vapor microchannel comprises a heterogenous catalyst;
heating the liquid in the wicking microchannel wherein oxygenate is stripped from the liquid phase and wherein at least one of the oxygenates is dehydrated over a hydrophobic solid acid catalyst in the vapor microchannel to produce water and a gaseous dehydrated product, and wherein the produced water preferentially passes back into the aqueous solution in the wick;
wherein the aqueous solution passes through the wick and out of the reaction chamber via a first liquid outlet; and
wherein the gaseous dehydrated product passes through the vapor microchannel and out of the reaction chamber through a vapor outlet.

7. The method of any of claims 5-6 wherein the liquid flowing through the wicking microchannel and the vapor flowing through the vapor microchannel are in counter flow.

8. The method of any of claims 5-6 wherein the liquid flowing through the wicking microchannel and the vapor flowing through the vapor microchannel are in co-flow.

9. The method of any of claims 5-8 wherein the mass average temperature of the liquid in the wick is at least 3° C. less, or at least 5° C. less, than the mass average temperature of the vapor in the vapor microchannel.

10. The method of any of claims 5-9 wherein the oxygenates comprise an alcohol.

11. The method of any of claims 5-10 wherein the oxygenates are at least 90 mass % of a single type of molecule.

12. The method of any of claims 5-10 wherein the oxygenates comprise at least 30 mass % of a first type of molecule and at least 30 mass % of a different second type of molecule.

13. The method of claim 12 wherein the first and second molecules are aldehydes or ketones that react via an aldol condensation.

14. The method of any of claims 5-13 wherein the solid acid catalyst comprises ZSM5.

15. The method of any of claims 6-13 wherein the wick comprises a metallic foam or metallic felt.

16. The method of any of claims 5-11 wherein the oxygenates are distilled from a fermenter and are passed into the reaction chamber without removing water prior to passing into the reaction chamber and in contact with the solid acid catalyst.

17. The method of any of claims 5-16 wherein the aqueous solution that passes out through the first liquid outlet is at least partly recycled to the reaction chamber.

18. The method of any of claims 5-17 wherein at least 20 mass %, or at least 30 mass %, or at least 40 mass %, or in the range of 20 to 55 mass %, of the oxygenates are converted in a single pass through the reaction chamber.

19. The method of any of claims 5-18 wherein the oxygenates are converted to a single product with a selectivity of at least 50% or at least 60%, or in the range of 50 to 80%.

20. The method of any of claims 18-19 wherein the reaction is conducted continuously and without catalyst regeneration or catalyst addition for at least 5 hours or at least 10 hours or for up to 12 hours, wherein the conversion and/or selectivity is maintained for the entire period.

21. (canceled)
